(19) United States
(12) Patent Application Publication
(10) Pub. No.: US 2017/0071928 A1
(43) Pub. Date: Mar. 16, 2017

(54) METHODS AND PHARMACEUTICAL COMPOSITIONS FOR TREATING DISEASES ASSOCIATED WITH ALTERED SERT ACTIVITY

(71) Applicant: THE TRUSTEES OF COLUMBIA UNIVESITY IN THE CITY OF NEW YORK, New York, NY (US)

(72) Inventors: Michael D. Gershon, New York, NY (US); Kara Gross Margolis, New York, NY (US)

(21) Appl. No.: 15/311,057

(22) PCT Filed: May 15, 2015
(86) PCT No.: PCT/US15/31184

§ 371 Date: Nov. 14, 2016

Related U.S. Application Data
(60) Provisional application No. 61/994,020, filed on May 15, 2014.

(51) Int. Cl. A61K 31/4525

(52) U.S. Cl. CPC A61K 31/4525

(57) ABSTRACT

The present invention provides, inter alia, methods for treating or ameliorating the effects of a disease associated with altered serotonin transporter molecule (SERT) activity in a subject in need thereof. The methods include administering to the subject an effective amount of a 5-HT₄ agonist or a pharmaceutically acceptable salt thereof. Pharmaceutical compositions and kits for the same are also provided. The present invention additionally provides methods for treating or ameliorating the effects of elevated serotonin transporter molecule activity in the gastrointestinal tract of a subject in need thereof and a gastrointestinal abnormality associated with autism spectrum disorder in a subject in need thereof.

### CROSS-REFERENCE TO RELATED APPLICATIONS

The present invention claims benefit to U.S. Provisional Application No. 61/994,020 filed May 15, 2014. The entire contents of the above application are incorporated by reference.

### GOVERNMENT FUNDING

This invention was made with government support under grant no. NS15547 and DK093786 awarded by the National Institutes of Health. The government has certain rights in the invention.

### FIELD OF INVENTION

The present invention provides, inter alia, methods for treating or ameliorating the effects of a disease associated with altered serotonin transporter molecule (SERT) activity, elevated serotonin transporter molecule activity in the gastrointestinal tract, and a gastrointestinal abnormality associated with autism spectrum disorder. Pharmaceutical compositions and kits are also provided.

### INCORPORATION BY REFERENCE OF SEQUENCE LISTING

This application contains references to amino acids and/or nucleic acid sequences that have been filed concurrently herewith as sequence listing text file “0384275.txt”, file size of 129 KB, created on May 15, 2015. The aforementioned sequence listing is hereby incorporated by reference in its entirety pursuant to 37 C.F.R. §1.52(e)(5).

### BACKGROUND OF THE INVENTION

Parents are often concerned about gastrointestinal (GI) symptoms in their children with autism spectrum disorder (ASD). Children with ASD experience significantly more general GI symptoms than comparison groups, with a standardized mean difference of 0.82 and a corresponding odds ratio (OR) of 4.42 (95% confidence interval (CI), 1.90-10.28). (McElhanon et al., 2014). These children also experience higher rates of constipation (OR: 3.86, 95% CI, 2.23-6.71) and diarrhea (OR: 3.63, 95% CI, 1.82-7.23). (Id.). However, systematic studies of the pathophysiology have been lacking and variable methods have precluded identifying organic bases of the problems. Thus, there exists an unmet need for methods and compositions suitable for treating or ameliorating the effects of ASD. This application is directed to, inter alia, meeting this and other needs.

### SUMMARY OF THE INVENTION

One embodiment of the present invention is a method for treating or ameliorating the effects of a disease associated with altered serotonin transporter molecule (SERT) activity in a subject in need thereof. The method comprises administering to the subject an effective amount of a 5-HT₄ agonist or a pharmaceutically acceptable salt thereof.

Another embodiment of the present invention is a method for treating or ameliorating the effects of elevated serotonin transporter molecule (SERT) activity in the gastrointestinal tract of a subject in need thereof. The method comprises administering to the subject an effective amount of a 5-HT₄ agonist or a pharmaceutically acceptable salt thereof.

A further embodiment of the present invention is a method for treating or ameliorating the effects of a gastrointestinal abnormality associated with autism spectrum disorder in a subject in need thereof. The method comprises administering to the subject an effective amount of a 5-HT₄ agonist or a pharmaceutically acceptable salt thereof.

An additional embodiment of the present invention is a pharmaceutical composition for treating or ameliorating the effects of a disease associated with altered serotonin transporter molecule (SERT) activity. The pharmaceutical composition comprises (i) a pharmaceutically acceptable diluent or carrier and (ii) an effective amount of a 5-HT₄ agonist or a pharmaceutically acceptable salt thereof.

Another embodiment of the present invention is a kit for treating or ameliorating the effects of a disease associated with altered serotonin transporter molecule (SERT) activity. The kit comprises an effective amount of a 5-HT₄ agonist, packaged together with instructions for its use.

### BRIEF DESCRIPTION OF THE DRAWINGS

FIG. 1 is a transmission electron micrograph showing that SERT in enterocytes (Ent) inactivates enterochromaffin (EC) cell 5-HT.

FIGS. 2A-2D show that total GI transit and colonic motility are slower in G56A mice than in wild-type (WT) mice. FIG. 2A is a histogram showing that total GI transit time was increased in G56A mice compared to WT mice. FIG. 2B is a histogram showing that colonic motility was slower in G56A mice compared to WT mice. FIG. 2C is a histogram showing that gastric emptying was not significantly different between G56A and WT mice. FIG. 2D is a histogram showing that the ability of exogenous 5-HT to accelerate small intestinal (SI) transit is blunted in G56A mice compared to WT mice.

FIGS. 3A-3C show that colonic migrating motor complexes (CMMCs) are abnormal in the isolated G56A colon. FIG. 3A is a box plot showing that CMMC frequency is less in G56A colon than in WT colon. FIG. 3B is a box plot showing that CMMC velocity is also less in G56A colon than in WT colon. FIG. 3C is a box plot showing that CMMC contraction length is less in G56A colon than in WT colon.

FIGS. 4A-4I show that total and late-born submucosal neurons are deficient in G56A mice. FIGS. 4A-4C are histograms showing that HuC/D neuronal protein-expressing cells (total neurons) (FIG. 4A), tyrosine hydroxylase (TH)-expressing cells (late born neurons) (FIG. 4B), and calcitonin gene-related peptide (CGRP)-expressing cells (late born neurons) (FIG. 4C) are decreased in G56A mice compared to WT mice. FIGS. 4D-4I are fluorescence micrographs showing HuC/D-expressing cells in WT (FIG. 4D) and G56A mice (FIG. 4E), TH-expressing cells in WT (FIG. 4F) and G56A mice (FIG. 4G), and CGRP-expressing cells in WT (FIG. 4H) and G56A mice (FIG. 4I).

FIGS. 5A-5F show that myenteric neurons are deficient in G56A mice. FIGS. 5A-5B are histograms showing that HuC/D neuronal protein-expressing cells are decreased in ileum (FIG. 5A) and colon (FIG. 5B) of G56A mice compared to WT mice. FIGS. 5C-5F are fluorescence micrographs showing decreased HuC/D-expressing cells in ileum of G56A mice (FIG. 5D) compared to WT mice (FIG. 5C) and in colon of G56A mice (FIG. 5F) compared to WT mice (FIG. 5E).

FIGS. 6A-6F also show that myenteric neurons are deficient in G56A mice. FIGS. 6A-6B are histograms showing that GABA-expressing cells are decreased in ileum (FIG. 6A) and colon (FIG. 6B) of G56A mice compared to WT mice. FIGS. 6C-6F are fluorescence micrographs showing decreased GABA-expressing cells in ileum of G56A mice (FIG. 6D) compared to WT mice (FIG. 6C) and in colon of G56A mice (FIG. 6F) compared to WT mice (FIG. 6E).

FIG. 7 is a histogram showing that the abundance of transcripts encoding CGRP is low in ilea of G56A mice.

FIG. 8 is a histogram showing that mortality from 2,4,6-trinitrobenzenesulfonic acid (TNBS)-induced colitis is greater in WT mice than in G56A mice.

FIGS. 9A-9D are line graphs showing that TNBS-induced colitis is more severe in WT mice than in G56A mice. Total clinical score (FIG. 9A), percent weight change (FIG. 9B), stool consistency (FIG. 9C), and stool blood (FIG. 9D) are decreased in G56A mice compared to WT mice.

FIGS. 10A-10B are histograms showing that expression of TNFα (FIG. 10A) and IL-1β (FIG. 10B) is greater in WT mice compared to G56A mice during TNBS-induced colitis.

FIGS. 11A-11C show that extra-enteric immune responses are similar in G56A mice and WT mice. Delayed hypersensitivity was assessed in the ear with dinitrofluorobenzene (DNFB). FIG. 11A is a histogram showing the percent change in ear thickness between DNFB-treated and control ears in WT and G56A mice. Transcripts encoding IL-1β and TNFα were measured in the treated vs the control ears. FIGS. 11B-11C are histograms showing IL-1β transcript ratios of DNFB-treated/control (FIG. 11B) and TNFα transcript ratios of DNFB-treated/control (FIG. 11C) in WT and G56A mice.

FIGS. 12A-12C show that bacterial load, small intestinal bacterial overload (SIBO), and invasion are greater in G56A mice than in WT mice. Ribosomal 16s (r16s) RNA was used to quantify bacterial load. FIG. 12A is a histogram showing ileum r16s levels normalized as percent colon in WT and G56A mice. FIGS. 12B-C are histograms showing r16s levels in mesenteric nodes (FIG. 12B) and stool (FIG. 12C) of WT and G56A mice.

FIGS. 13A-13C are transmission electron micrographs showing that more bacteria exist in the small intestines of G56A mice than in those of WT mice.

FIG. 14 is a histogram showing that intestinal permeability in WT mice is greater than in G56A mice. Data shown is baseline and post-dextran sodium sulfate (post-DSS) treatment.

FIGS. 15A-15B are transmission electron micrographs showing that transepithelial transit of horseradish peroxidase (HRP) is blocked at tight junctions in G56A and WT mice.

FIGS. 16A-16B are histograms showing that TPH1 expression is elevated (FIG. 16A), but TPH2 expression is decreased (FIG. 16B) in G56A mice compared to WT mice.

FIGS. 17A-17G show that villus height, crypt depth, and proliferation are lower in G56A mice compared to WT mice. FIGS. 17A-17B are light micrographs showing WT (FIG. 17A) and G56A (FIG. 17B) villus height. FIGS. 17C-17D are histograms showing that proliferation, as shown by the number of cells having the ki67⁺ marker per crypt, is lower in G56A mice than in WT mice in both the small intestine (FIG. 17C) and the colon (FIG. 17D). FIGS. 17E-17G are histograms showing villus height (FIG. 17E) and crypt depth (FIG. 17F) in small intestine and crypt depth in colon (FIG. 17G) of WT and G56A mice.

### DETAILED DESCRIPTION OF THE INVENTION

One embodiment of the present invention is a method for treating or ameliorating the effects of a disease associated with altered serotonin transporter molecule (SERT) activity in a subject in need thereof. The method comprises administering to the subject an effective amount of a 5-HT₄ agonist or a pharmaceutically acceptable salt thereof.

As used herein, the terms “treat,” “treating,” “treatment” and grammatical variations thereof mean subjecting an individual subject (e.g., a human patient) to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, “serotonin transporter molecule (SERT)” refers to the protein encoded by the solute carrier family 6 (neurotransmitter transporter; serotonin, member 4) (SLC6A4) gene. Its target, serotonin or, 5-hydroxytryptamine (5-HT), is synthesized from L-tryptophan via a series of enzymatic hydroxylation and decarboxylation reactions. Serotonin may be synthesized in the gut by enteric neurons and enterochromaffin cells to modify digestive processes such as intestinal transport and motility, as well as proliferation of gastrointestinal epithelium. Once released from serotonergic neurons, serotonin may bind to postsynaptic 5-HT receptors to effect downstream intracellular signaling pathways. (Rose′Meyer, 2013).

Tables 1 and 2 show the SEQ ID Nos. of representative nucleic acid and amino acid sequences of 5-HT₄ receptor and SERT, respectively, from various animals.

TABLE 1
5-HT₄ Receptor Sequences

| SEQ ID | | | Nucleotide/ | Additional |
|---|---|---|---|---|
| NO. | Organism | Gene Name | Polypeptide | Information |
| 1 | Homo | 5-hydroxytryptamine | Nucleotide | Variant a |
| | sapiens | receptor 4 | | |
| 2 | Homo | 5-hydroxytryptamine | Nucleotide | Variant b |
| | sapiens | receptor 4 | | |
| 3 | Homo | 5-hydroxytryptamine | Nucleotide | Variant c |
| | sapiens | receptor 4 | | |
| 4 | Homo | 5-hydroxytryptamine | Nucleotide | Variant d |
| | sapiens | receptor 4 | | |
| 5 | Homo | 5-hydroxytryptamine | Nucleotide | Variant g |
| | sapiens | receptor 4 | | |
| 6 | Homo | 5-hydroxytryptamine | Nucleotide | Variant i |
| | sapiens | receptor 4 | | |
| 7 | Mus | 5-hydroxytryptamine | Nucleotide | |
| | musculus | receptor 4 | | |
| 8 | Homo | 5-hydroxytryptamine | Polypeptide | Isoform a |
| | sapiens | receptor 4 | | |
| 9 | Homo | 5-hydroxytryptamine | Polypeptide | Isoform b |
| | sapiens | receptor 4 | | |
| 10 | Homo | 5-hydroxytryptamine | Polypeptide | Isoform c |
| | sapiens | receptor 4 | | |
| 11 | Homo | 5-hydroxytryptamine | Polypeptide | Isoform d |
| | sapiens | receptor 4 | | |
| 12 | Homo | 5-hydroxytryptamine | Polypeptide | Isoform g |
| | sapiens | receptor 4 | | |
| 13 | Homo | 5-hydroxytryptamine | Polypeptide | Isoform i |
| | sapiens | receptor 4 | | |
| 14 | Mus | 5-hydroxytryptamine | Polypeptide | |
| | musculus | receptor 4 | | |

TABLE 2
SERT Sequences

| SEQ ID | | | Nucleotide/ | Additional |
|---|---|---|---|---|
| NO. | Organism | Gene Name | Polypeptide | Information |
| 15 | Homo | Solute carrier family | Nucleotide | NM_001045.5 |
| | sapiens | 6 (neurotransmitter | | |
| | | transporter), | | |
| | | membe r4 (SLC6A4) | | |
| 16 | Homo | Sodium-dependent | Polypeptide | NP_001036.1 |
| | sapiens | serotonin transporter | | |
| 17 | Mus | Solute carrier family | Nucleotide | NM_010484.2 |
| | musculus | 6 (neurotransmitter | | |
| | | transporter, | | |
| | | serotonin), | | |
| | | member 4 (Slc6a4) | | |
| 18 | Mus | Sodium-dependent | Polypeptide | NP_034614.2 |
| | musculus | serotonin transporter | | |
| 19 | Rattus | Solute carrier family | Nucleotide | NM_013034.4 |
| | norvegicus | 6 (neurotransmitter | | |
| | | transporter), | | |
| | | member 4 (Slc6a4) | | |
| 20 | Rattus | Sodium-dependent | Polypeptide | NP_037166.2 |
| | norvegicus | serotonin transporter | | |
| 21 | Gallus | Solute carrier family | Nucleotide | NM_213572.1 |
| | gallus | 6 (neurotransmitter | | |
| | | transporter, | | |
| | | serotonin), | | |
| | | member 4 (SLC6A4) | | |
| 22 | Gallus | Sodium-dependent | Polypeptide | NP_998737.1 |
| | gallus | serotonin transporter | | |
| 23 | Canis lupus | Solute carrier family | Nucleotide | NM_001110771.1 |
| | familiaris | 6 (neurotransmitter | | |
| | | transporter, | | |
| | | serotonin), | | |
| | | member 4 (SLC6A4) | | |
| 24 | Canis lupus | Sodium-dependent | Polypeptide | NP_001104241.1 |
| | familiaris | serotonin transporter | | |
| 25 | Bos taurus | Solute carrier family | Nucleotide | NM_174609.2 |
| | | 6 (neurotransmitter | | |
| | | transporter, | | |
| | | serotonin), | | |
| | | member 4 (SLC6A4) | | |
| 26 | Bos taurus | Sodium-dependent | Polypeptide | NP_777034.1 |
| | | serotonin transporter | | |
| 27 | Cavia | Solute carrier family | Nucleotide | NM_001173018.1 |
| | porcellus | 6 (neurotransmitter | | |
| | | transporter, | | |
| | | serotonin), | | |
| | | member 4 (Slc6a4) | | |
| 28 | Cavia | Sodium-dependent | Polypeptide | NP_001166489.1 |
| | porcellus | serotonin transporter | | |

SERT transports serotonin from the synaptic space into presynaptic neurons, thereby terminating serotonin's effects. SERT activity is related to its phosphorylation state; SERT may be phosphorylated by a number of kinases including protein kinase C (PKC), protein kinase G (PKG), and p38 mitogen-activated protein kinase (MAPK). Cytokines released in the inflammatory process, such as IL-10, IFN-γ, and TNF-α, may also control SERT activity. When SERT expression increases or SERT activity is enhanced, serotonin is more efficiently removed from the synaptic cleft. (Id.).

As used herein, “SERT activity” refers to the removal of serotonin from the synaptic space into presynaptic neurons by the serotonin transporter protein. The term “altered”, in reference to SERT activity refers to any SERT activity that deviates from that observed in a given tissue in a wild-type specimen, such as increased activity or decreased activity in a given tissue, e.g., the gastrointestinal tract of a subject.

SERT activity may be determined via any procedure or combination of procedures, including, but not limited to, Western blotting for total and/or phosphorylated SERT levels in a given tissue with optional exposure to various agents such as, but not limited to, 8-bromo-cGMP (an activator of PKG) and PD169316 (an inhibitor of p38 MAPK), in vivo chronoamperometry to measure serotonin clearance, measurement of a stereotyped head twitch response, and determination of hypothermia sensitivity. (Veenstra-VanderWeele et al., 2012).

Western blotting is a protocol well known to those of skill in the art. In brief, chronoamperometry is a square wave pulsed voltammetric technique. Chronoamperometry generates high charging currents which decay exponentially with time. To measure the faradic current (the current that is proportional to the concentration of the analyte, i.e. serotonin), current in the last 70-80% of each scan is integrated (when charging current has dissipated).

Another method for assessing SERT activity utilizes the 5-HT_{2A/2C} receptor agonist 1-(2,5-dimethoxy-4-iodophenyl)-2-aminopropane (DOI), which induces a stereotyped head twitch response mediated by postsynaptic, cortical 5-HT_{2A} receptors that can be measured as twitches/unit time. Additionally, the 5-HT_{1A/7} agonist 8-hydroxy-2-(di-n-propylamino)-tetraline (8-OH-DPAT) causes hypothermia in mice mediated by 5-HT_{1A} receptors. Therefore, routine temperature measurements can be used to determine sensitivity to 8-OH-DPAT.

Thus, “increased” SERT activity in a subject with a disease compared to a subject that does not have the disease may be indicated by higher levels of total/phosphorylated SERT in a given tissue, faster serotonin clearance rates, more head twitches in a set period of time upon exposure to DOI, larger temperature decreases upon exposure to 8-OH-DPAT, and combinations thereof in a subject with a disease compared to a subject without the disease. Likewise, “increased” or “elevated” SERT activity in the gastrointestinal tract in a subject with a disease may be determined by a number of techniques similar to those discussed above utilizing GI tissues, such as, for example, probing GI tissue lysates for phosphorylated/total SERT levels using Western blotting techniques.

As used herein, a disease associated with altered SERT includes, but is not limited to, autism spectrum disorder, attention deficit hyperactivity disorder (ADHD), bipolar disorder, Tourette's syndrome, chronic intestinal pseudoobstruction (CIP), functional gastrointestinal disorders (FGID), irritable bowel syndrome (IBS), chronic constipation, functional dyspepsia, and combinations thereof. In a preferred aspect of this embodiment, the disease is autism spectrum disorder.

As used herein, a “subject” is a mammal, preferably, a human. In addition to humans, categories of mammals within the scope of the present invention include, for example, primates, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

As used herein, a “5-HT₄ agonist” is any molecule that activates the 5-HT₄ receptor, and includes, but is not limited to, BIMU-8, cisapride, CJ-033,466, ML-10302, mosapride, prucalopride, renzapride, RS-67506, RS-67333, SL65.0155, tegaserod, zacopride, ATI-7505, velusetrag (TD-5108), levosulpiride, cinitapride, metoclopramide, pharmaceutically acceptable salts thereof, and combinations thereof. Preferably, the 5-HT₄ agonist is prucalopride or a pharmaceutically acceptable salt thereof.

In another aspect of this embodiment, the subject has a mutation in a gene encoding SERT. As used herein, a “mutation” may be a substitution (e.g., a point mutation), deletion, insertion, translocation, inversion, or a fusion. Methods for identifying mutations in nucleic acids, such as the above-listed SLC6A4 genes, are known in the art. Non-limiting examples include PCR, sequencing, hybrid capture, in-solution capture, molecular inversion probes, fluorescent in situ hybridization (FISH) assays, and combinations thereof.

Various sequencing methods are known in the art. These include, but are not limited to, Sanger sequencing (also referred to as dideoxy sequencing) and various sequencing-by-synthesis (SBS) methods as disclosed in, e.g., Metzker 2005, sequencing by hybridization, by ligation (for example, WO 2005021786), by degradation (for example, U.S. Pat. Nos. 5,622,824 and 6,140,053) and nanopore sequencing (which is commercially available from Oxford Nanopore Technologies, UK). In deep sequencing techniques, a given nucleotide in the sequence is read more than once during the sequencing process. Deep sequencing techniques are disclosed in e.g., U.S. Patent Publication No. 20120264632 and International Patent Publication No. WO2012125848.

PCR-based methods for detecting mutations are known in the art and employ PCR amplification, where each target sequence in the sample has a corresponding pair of unique, sequence-specific primers. For example, the polymerase chain reaction-restriction fragment length polymorphism (PCR-RFLP) method allows for rapid detection of mutations after the genomic sequences are amplified by PCR. The mutation is discriminated by digestion with specific restriction endonucleases and is identified by electrophoresis. See, e.g., Ota et al., 2007. Mutations may also be detected using real time PCR. See, e.g., International Application publication No. WO2012046981.

Hybrid capture methods are known in the art and are disclosed in e.g., U.S. Patent Publication No. 20130203632 and U.S. Pat. Nos. 8,389,219 and 8,288,520. These methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g. biotin) which can subsequently be immobilized to a solid surface, such as a bead (in-solution capture).

Molecular Inversion Probe (MIP) techniques are known in the art and are disclosed in e.g., Absalan et al., 2008. This method uses MIP molecules, which are special “padlock” probes (Nilsson et al., 1994) for genotyping. A MIP molecule is a linear oligonucleotide that contains specific regions, universal sequences, restriction sites and a Tag (index) sequence (16-22 bp). A MIP hybridizes directly around the genetic marker/SNP of interest. The MIP method may also use a number of “padlock” probe sets that hybridize to genomic DNA in parallel (Hardenbol et al., 2003). In case of a perfect match, genomic homology regions are ligated by undergoing an inversion in configuration (as suggested by the name of the technique) and creating a circular molecule. After the first restriction, all molecules are amplified with universal primers. Amplicons are restricted again to ensure short fragments for hybridization on a microarray. Generated short fragments are labeled and, through a Tag sequence, hybridized to a cTag (complementary strand for index) on an array. After the formation of Tag-cTag duplex, a signal is detected.

In a preferred aspect of this embodiment, the mutation is a G56A mutation in the gene encoding SERT. The G56A mutation in SERT refers to the substitution of glycine with alanine at sequence position corresponding to amino acid number 56 of mouse or human SERT. G56A SERT exhibits increased basal phosphorylation and leads to increased serotonin trafficking, thereby decreasing the exposure time of postsynaptic neurons to serotonin. Notably, The G56A SERT variant has a higher prevalence in individuals with ASD. (Rose′Meyer, 2013). Though transgenic mice expressing G56A SERT exhibited normal growth patterns and fertility, these mice also showed increased CNS serotonin clearance and serotonin receptor sensitivity, as well as hyperserotonemia. (Id.)

Another embodiment of the present invention is a method for treating or ameliorating the effects of elevated serotonin transporter molecule (SERT) activity in the gastrointestinal tract of a subject in need thereof. The method comprises administering to the subject an effective amount of a 5-HT₄ agonist or a pharmaceutically acceptable salt thereof.

In the present embodiment, suitable and preferred 5-HT₄ agonists, subjects, and diseases are as set forth above.

A further embodiment of the present invention is a method for treating or ameliorating the effects of a gastrointestinal abnormality associated with autism spectrum disorder in a subject in need thereof. The method comprises administering to the subject an effective amount of a 5-HT₄ agonist or a pharmaceutically acceptable salt thereof.

As used herein, gastrointestinal abnormalities associated with autism spectrum disorder include, but are not limited to, enteric development defects, chronic diarrhea, chronic constipation, irritable and/or inflammatory bowel syndromes (e.g. colitis), and gastroesophageal reflux disease (GERD).

An additional embodiment of the present invention is a pharmaceutical composition for treating or ameliorating the effects of a disease associated with altered serotonin transporter molecule (SERT) activity. The pharmaceutical composition comprises (i) a pharmaceutically acceptable diluent or carrier and (ii) an effective amount of a 5-HT₄ agonist or a pharmaceutically acceptable salt thereof.

In this embodiment, suitable and preferred 5-HT₄ agonists and diseases associated with altered SERT activity are as set forth above.

Another embodiment of the present invention is a kit for treating or ameliorating the effects of a disease associated with altered serotonin transporter molecule (SERT) activity. The kit comprises an effective amount of a 5-HT₄ agonist, packaged together with instructions for its use.

In this embodiment, suitable and preferred 5-HT₄ agonists and diseases associated with altered SERT activity are as set forth above.

The kits may also include suitable storage containers, e.g., ampules, vials, tubes, etc., for each 5-HT₄ agonist of the present invention and other reagents, e.g., buffers, balanced salt solutions, etc., for use in administering the 5-HT₄ agonists to subjects. The 5-HT₄ agonists may be present in the kits in any convenient form, such as, e.g., in a solution or in a powder form. The kits may further include a packaging container, optionally having one or more partitions for housing the 5-HT₄ agonists and other optional reagents.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a compound or composition according to the invention will be that amount of the composition which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of 5-HT₄ agonists disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of 5-HT₄ agonists disclosed herein, may be administered as two, three, four, five, six or more sub-doses, administered separately at appropriate intervals throughout the day.

The 5-HT₄ agonists or a pharmaceutical composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, 5-HT₄ agonists or a pharmaceutical composition of the present invention may be administered in conjunction with other treatments. 5-HT₄ agonists or a pharmaceutical composition of the present invention may be encapsulated or otherwise protected against gastric or other secretions, if desired.

The pharmaceutical compositions of the invention comprise one or more active ingredients in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable diluents or carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

Any formulation of the invention may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The following examples are provided to further illustrate the methods of the present invention. These examples are illustrative only and are not intended to limit the scope of the invention in any way.

### EXAMPLES

### Example 1

### Enteric 5-HT is a Multifunctional Signaling Molecule

5-HT has been postulated to play roles in regulating GI motility (tryptophan hydroxylase 1 (TPH1) and TPH2), promoting development of enteric neurons (TPH2), initiating adult neurogenesis (TPH1), mucosal maintenance (TPH2), promoting inflammation (TPH1), protecting against bacterial overgrowth and invasion (TPH1), and regulating bone formation and metabolism (TPH1).

A transgenic mutant mouse line was generated in which the defect was in the serotonin transporter molecule (SERT), which is also the target of antidepressants, such as serotonin selective reuptake inhibitors (SSRIs). The SERT mutation expressed in this mouse was a substitution of one amino acid at position 56, G56A (G=glycine; A=alanine). The result of the mutation was that the molecule became post-translationally modified and excessively active. The mutated molecule clears serotonin away from its receptors (5-HT receptors) before serotonin can adequately stimulate those receptors. This effect caused the functions of serotonin to become deficient. (Veenstra-VanderWeele et al., 2012).

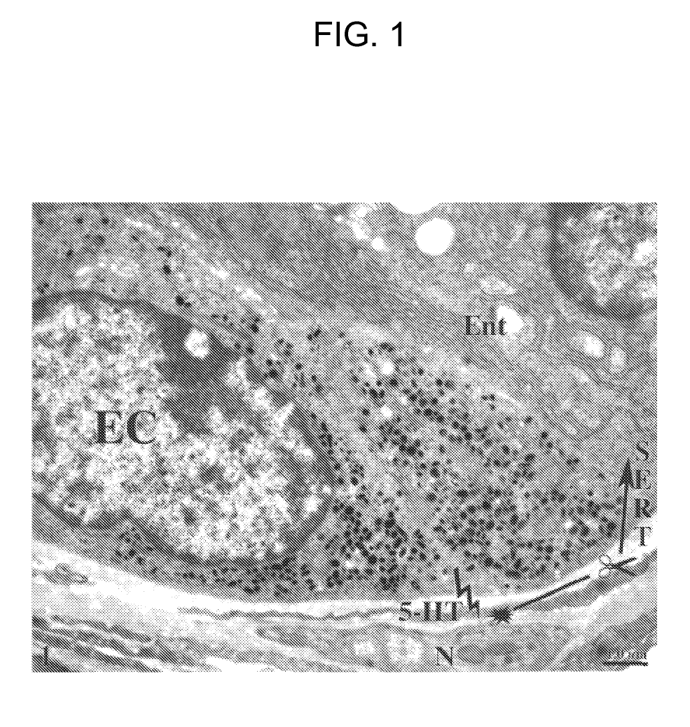

G56A is the most common gain-of-function SERT coding variant in children on the autistic spectrum. 5-HT receptor inactivation is SERT-dependent: increases in SERT activity decrease the effects of 5-HT on 5-HT receptors as is observed in enterocytes (FIG. 1), while deletion of SERT increases the effects of 5-HT. G56A results in constitutive p38 MAPK-dependent phosphorylation of SERT and enhanced 5-HT clearance, leading to hyperserotonemia (increased platelet 5-HT). Accompanying brain/behavioral abnormalities result as well, including altered basal firing of serotonergic raphe neurons, 5HT_{1A} and 5HT_{2A} receptor hypersensitivity, and autism-like altered social function, characterized by communication deficits and repetitive behaviors.

### Example 2

### Materials and Methods

Carmine red, which cannot be absorbed from the lumen of the gut, was used to study total GI transit time (Kimball et al., 2005). A solution of carmine red (300 μl; 6%; Sigma Aldrich, St. Louis, Mo.) suspended in 0.5% methylcellulose (Sigma Aldrich, St. Louis, Mo.) was administered by gavage through a 21-gauge round-tip feeding needle. The time at which gavage took place was recorded as TO. After gavage, fecal pellets were monitored at 10 minutes intervals for the presence of carmine red. Total GI transit time was considered as the interval between TO and the time of first observance of carmine red in stool.

Colonic motility was studied as previously described (Li et al., 2006). Briefly, the animals were anesthetized with isoflurane (Baxter Pharmaceutical Products Inc, Deerfield, Ill.). A glass bead (3 mm in diameter) was pushed into the colon to a distance of 2 cm from the anal verge. The time required for expulsion of the glass bead was measured and taken as an estimate of colonic motility.

For analysis of gastric emptying and small intestinal transit, mice were fasted overnight in cages that lacked bedding. Water was withdrawn 3 hours before the experiment. A solution containing rhodamine B dextran (100 μl; 10 mg/ml in 2% methylcellulose, Invitrogen, Carlsbad, Calif.) was administered to each mouse by gavage through a 21-gauge round-tip feeding needle. Animals were euthanized 15 minutes after gavage; the stomach, small intestine, cecum, and colon were collected in 0.9% NaCl. The small intestine was divided into 10 segments of equal length and the colon (used to obtain total recovered rhodamine B fluorescence) was divided in half. Each piece of tissue was then transferred into a 14-ml tube containing 4 ml of 0.9% NaCl, homogenized, and centrifuged (2,000×g) to obtain a clear supernatant. Rhodamine fluorescence was measured in 1 ml aliquots of the supernatant (VersaFluor™ Fluorometer; BIO-RAD Laboratories, Hercules, Calif.). The proportion of the Rhodamine B dextran that emptied from the stomach was calculated as [(total recovered fluorescence−fluorescence remaining in the stomach)/(total recovered fluorescence)]×100.

Small intestinal transit was estimated by the position of the geometric center of the Rhodamine B dextran in the small bowel (Miller et al., 1981). For each segment of the small intestine (1-10), the geometric center (a) was calculated as: a=(fluorescence in each segment×number of the segment)/(total fluorescence recovered in the small intestine). The total geometric center=Σ (a of each segment). Total geometric center values are distributed between 1 (minimal motility) and 10 (maximal motility).

Colonic migrating motor complexes (CMMCs) were measured in isolated preparations of jejunum (5-7 cm) and colon (full-length) in parallel organ baths. Each segment was cannulated at the oral and anal ends to permit luminal pressure to be controlled and drugs to be administered. After equilibration, 4×15 minute control videos were recorded. Compounds, such as tetrodotoxin (TTX), to verify that observed activity was neuronal in origin, were then given (in the superfusate and/or intraluminally) and documented with 4×15 minute videos. The drug was then washed out and 4×15 minute videos were again obtained. This protocol allowed CMMCs to be compared in WT and G56A animals±TTX or appropriate agonists and antagonists. The drug washouts served as tests for time-dependent changes in baseline and reversibility of drugs. Up to 10 repetitions (animals) for WT and mutant mice enabled significant quantitative differences in the CMMC parameters of frequency, length propagated, propagation speed, and maximum contraction to be detected.

Immunocytochemistry was performed as follows: HuC/D, TH, g-aminobutyric acid (GABA), nNOS, and CGRP were located immunocytochemically to determine the abundance of total, dopaminergic, GABAergic, nitrergic and CGRP-expressing enteric neurons in G56A mice and their respective WT littermates. SERT immunoreactivity was also investigated to determine whether or not it is coincident with TH immunoreactivity in the myenteric plexus. In each experimental group, tissue samples were collected from ilea of 6-8 animals, fixed with 4% formaldehyde for 1.5 hours and washed in PBS. Whole mount preparations of longitudinal muscle with attached myenteric plexus (LMMP) were prepared by dissection. Cultured cells were fixed with 4% formaldehyde for 30 minutes and washed in PBS. Preparations were blocked with 10% normal horse serum for 30 minutes at room temp and then incubated for 48 or 72 hours at 4° C. with primary antibodies. Bound primary antibodies were visualized, respectively, with streptavidin-Alexa 594 for mouse monoclonal antibodies, and donkey antibodies to goat, rabbit, or sheep IgG coupled to Alexa 350, 488 or 594 (diluted 1:200; Invitrogen). Preparations were washed with PBS, mounted in buffered glycerol, and examined with a Leica CTR 6000 microscope. Images were obtained with a cooled CCD camera and analyzed with computer assistance (Volocity 5.4 imaging software; Improvision, Waltham, Mass.). To count the numbers of HuC/D-immunoreactive cells in each LMMP preparation, a motorized stage under computer control was used to scan and collect images with a 40× objective covering the entirety of a 10 mm² area. To count the numbers of CGRP-, nNOS- and TH-immunoreactive cells a similar procedure was used, except that images were collected with a 20× objective and that an area of 30 mm² was scanned. The higher magnification was needed for HuC/D-immunoreactive cells to be certain that overlapping cells could be distinguished. The lower magnification was advantageous for TH-immunoreactive cells, which are widely separated and relatively rare, because the cells were scattered and large numbers could be imaged for statistical reliability. The collected images were processed using a computer running Volocity 5.6 software to determine the numbers of immunoreactive cells of each type, which were presented as cells/mm². TH-immunoreactive terminal axons in the myenteric plexus of ileum of four G56A SERT and four WT mice were examined to study the effects of G56A SERT on the density of extrinsic sympathetic terminals. Sympathetic axons are far more abundant that dopaminergic axons in the myenteric plexus. Their large varicosities and coarse structure also distinguish sympathetic from dopaminergic axons. Dopaminergic neurons, moreover, are primarily located in the submucosal plexus. In each preparation, 3 images were obtained with a 40× objective. The total area occupied by TH-immunoreactive terminals in each image was obtained with computer assistance (Volocity 5.6 software; Improvision, Perkin Elmer).

CGRP, GAPDH, r16S, TPH1, and TPH2 RNA expression levels were determined using quantitative reverse transcriptase polymerase chain reaction. Briefly, transcripts encoding CGRP, GAPDH, r16S, TPH1, and TPH2 were quantified to evaluate the amount of said transcripts in the ilea. Transcripts encoding molecules in inflammatory pathways (TNF-α, and IL-1β) were also quantified to assess the severity of inflammation in G56A and WT littermates. RNA was extracted with Trizol (Invitrogen) and treated with DNase I (1 U·ml⁻¹). PCR, utilizing primers for β-actin, confirmed absence of DNA contamination. Reverse transcriptase (High Capacity cDNA Archive Kit; Applied Biosystems) was used to convert 1 μg of sample to cDNA. RT-PCR was employed to quantify transcripts encoding TNF-α, and IL-1β. For all non-GAPDH-encoding transcripts, expression was normalized to glyceraldehyde 3-phosphate dehydrogenase (GAPDH). The real-time reaction contained cDNA (5 μl), primers (Applied Biosystems; Foster City, Calif.) for the cytokine/chemokine/standard (250 nmol), PCR master mix (12.5 μl; Applied Biosystems) and nuclease-free water (6.25 μl). A GeneAmp 7500 sequence detection system (Applied Biosystems) was used to quantify cDNA levels. Duplicates were incubated for 2 minutes at 50° C., denatured for 10 minutes at 95° C., and subjected to 40 cycles of annealing at 60° C. for 20 seconds, extension at 60° C. for 1 minute, and denaturation at 95° C. for 15 sec. TaqMan 7500 software was used for data analysis.

Experimental colitis was induced with 2,4,6-trinitrobenzenesulfonic acid (TNBS; administered rectally; colons examined after 7 days) or dextran sodium sulfate (DSS; administered orally for 5-6 days in drinking water). TNBS (100 μl; 100 mg/kg) or saline (control) in 30% ethanol was infused into the colonic lumen 3.5 cm from the anal verge via a polyethylene cannula affixed to a 1-ml syringe. Alternatively, colitis was induced with DSS (5% in drinking water; 5-6 days) (Margolis et al., 2011). Persistent weight loss and loose blood-containing stools identified colitis. A clinical disease activity index was computed daily based on changes in body weight (5-point scale), stool consistency (3-point scale), and blood in stools (3-point scale). (Id.). Colons were removed from euthanized mice 7 days following TNBS infusion. In addition to clinical scores, histological examination of stained sections of paraffin-embedded distal colon was employed to evaluate severity of colitis. Transverse sections (5 μM) of paraffin-embedded distal colon (3 cm) were stained with hematoxylin and eosin. An animal pathologist, blinded to each animal's treatment, assigned histological scores. For inflammation, the score was: 0 when only rare inflammatory cells were present in the lamina propria, 1 when increased numbers of granulocytes were present in the lamina propria, 2 when inflammatory cells became confluent in the mucosa and extended into the submucosa, 3 when the inflammatory infiltrate extended across the intestinal wall. For crypt damage, the score was: 0 when crypts were intact, 1 when the basal third of crypts were lost, 2 when the basal two thirds of crypts were lost, 3 when entire crypts were lost, 4 when the epithelial surface was changed and erosions were observed, 5 when the epithelial surface was completely eroded. For evaluation of ulcers, the score was 0 when ulceration was absent, 1 when one or two foci of ulcerations were evident, 2 when 3 foci of ulceration were observed, 3 when ulcers were confluent and/or extensive. Severity was estimated from the sum of the individual scores.

In vivo permeability was determined as follows: the absorption of fluorescein isothiocyanate (FITC) labeled dextran was evaluated by measuring its concentration in blood after administration by oral gavage. FITC-dextran (4.4 kDa; 22 mg·ml⁻¹ in PBS; pH 7.4; Sigma, St Louis, Mich.) was administered orally by gavage. Blood samples (100 μl) were obtained from a submandibular vein 2 and 5 hours after the administration of FITC-dextran from WT (n=6) and G56A mice (n=6). The fluorescein concentration was determined by measuring fluorescence at 520 nm.

Villi experiments were performed as follows: segments of colon and small intestine were fixed for 3 hours at room temperature with 4% formaldehyde (from paraformaldehyde) and 2.5% glutaraldehyde in 0.1M phosphate buffer (pH 7.4) containing 3.5% sucrose and post-fixed with 1% OsO₄ for 1 hour. Fixed tissue was washed, dehydrated with ethanol, cleared in propylene oxide, and embedded in Spurr's low viscosity resin. Sections were cut at 0.9 μm and stained with toluidine blue to measure villus height (VH; small intestine) and crypt depth (CD; small intestine and colon) at 1000× magnification with computer-assisted imaging (Volocity 4.0; Perking Elmer, Waltham, Mass.). Villi (20/mouse) were measured when the central lacteal was completely visualized. Crypts (20/mouse) were analyzed when the crypt-villus junction could be visualized on both sides of the crypt.

All other imaging was performed using transmission electron microscopy (TEM). Preparations were similar to the villi experiments, except that sections are cut, prepared for TEM, and examined with an electron microscope, rather than a light microscope. Sections were cut at 0.6 nm and either counterstained with uranyl acetate and lead citrate or left unstained to visualize DAB. Sections were examined with a JEOL 1200EX electron microscope.

### Example 3

### Effects of G56A SERT on Mice

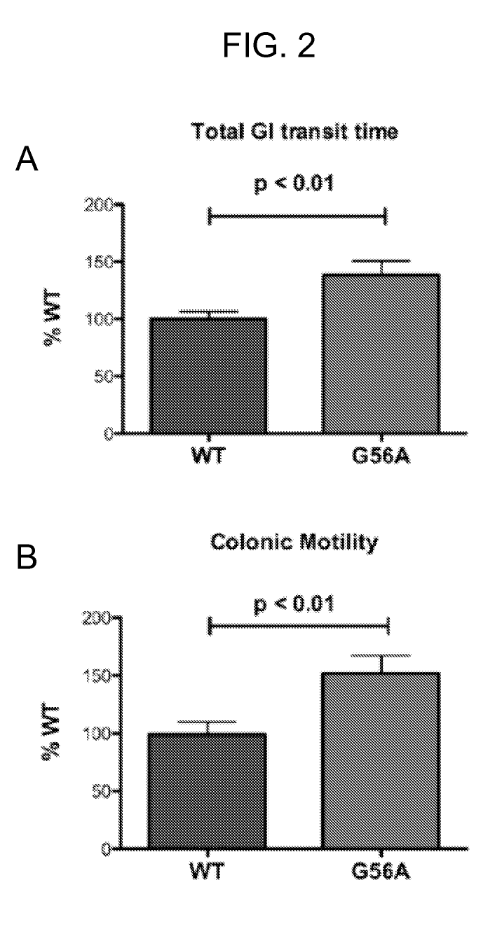

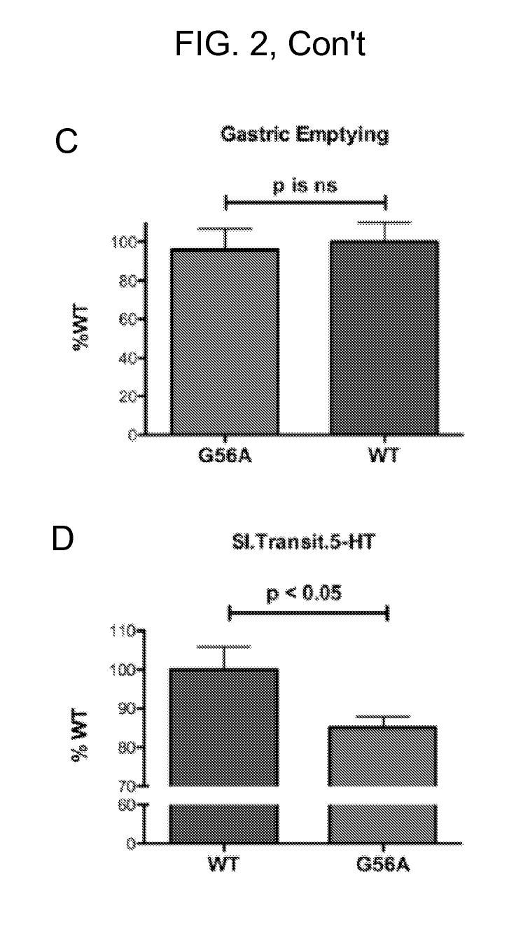

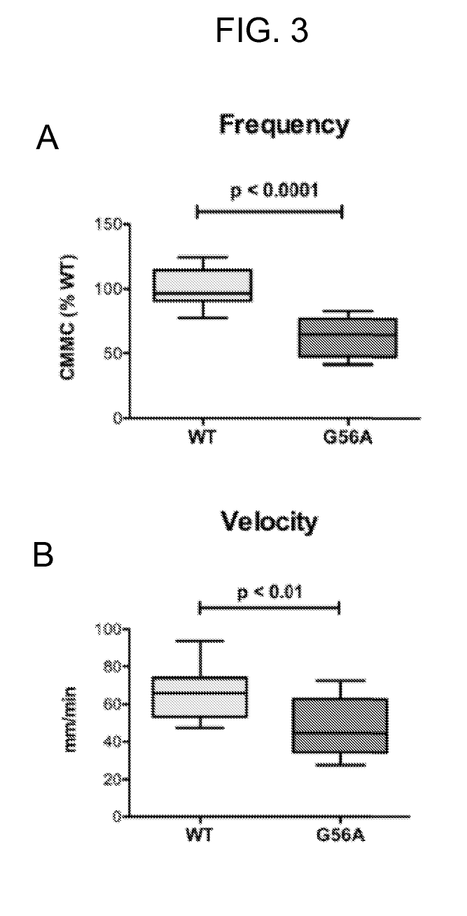

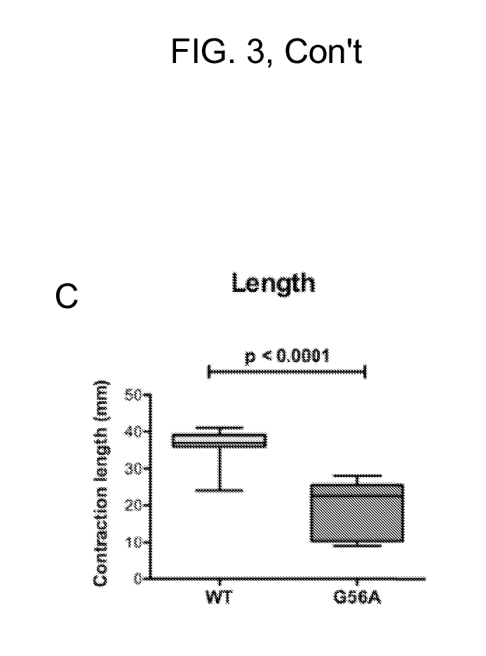

Total GI transit time and colonic motility were slower in G56A mice compared to WT mice (FIGS. 2A-2B), while no significant difference in gastric emptying was observed (FIG. 2C). The ability of exogenous 5-HT to accelerate small intestinal transit was also impaired in G56A mice (FIG. 2D). Additionally, CMMC frequency (FIG. 3A), velocity (FIG. 3B), and length (FIG. 3C) were all less in G56A than in WT colon, indicating that enteric nervous system (ENS) regulation of peristaltic activity is defective in G56A mice.

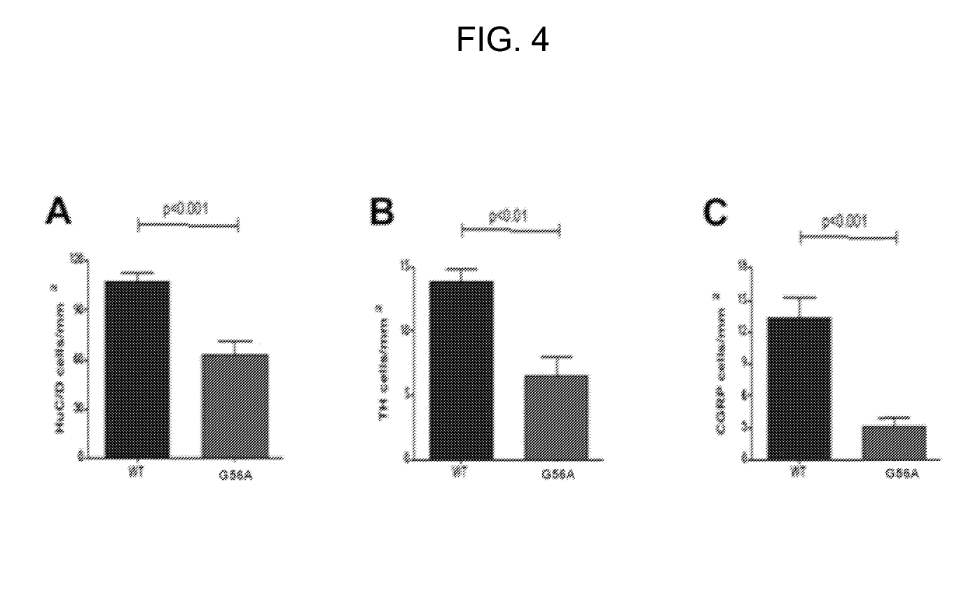

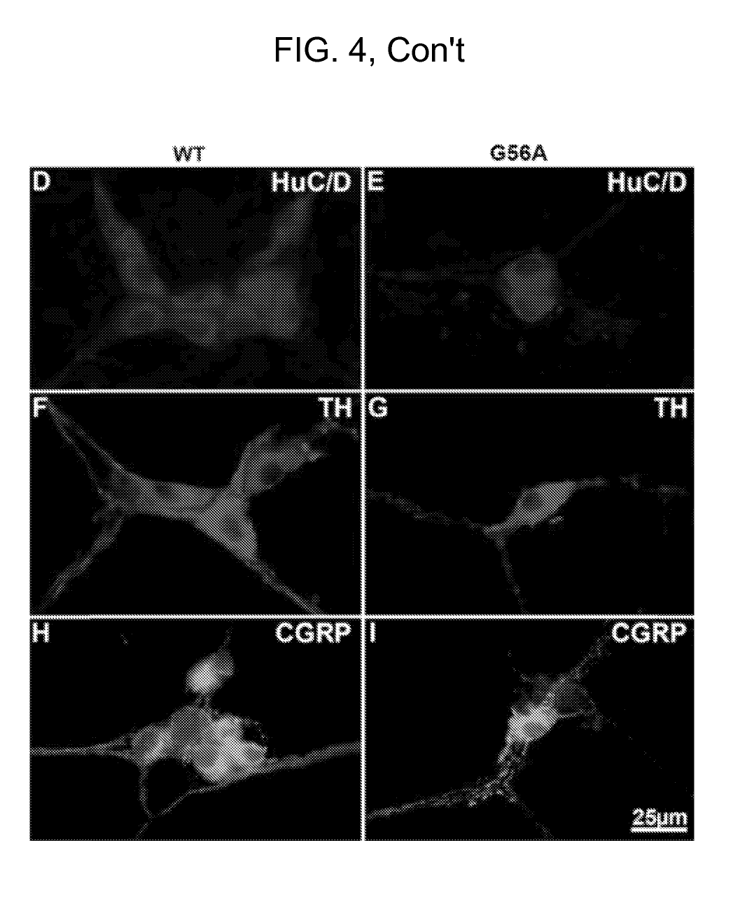

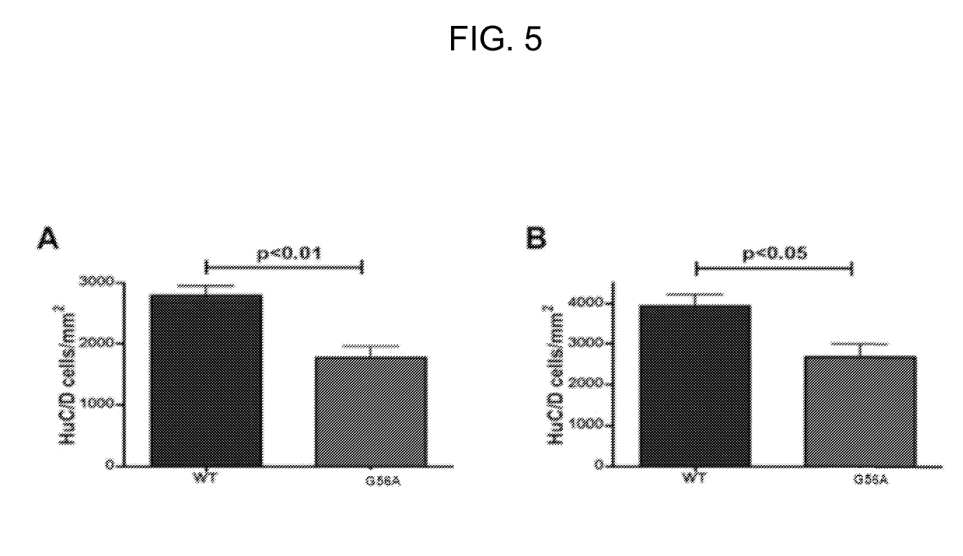

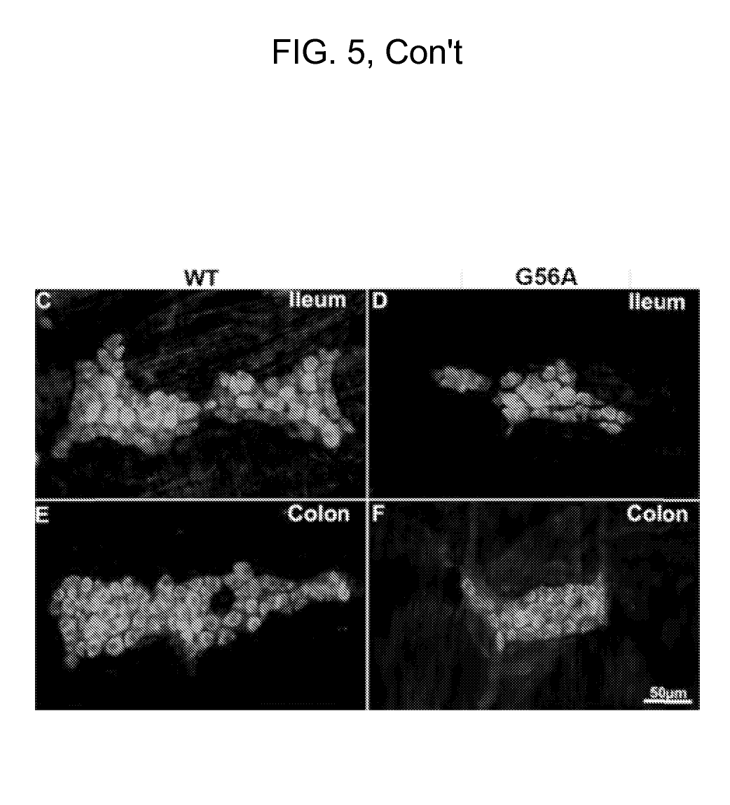

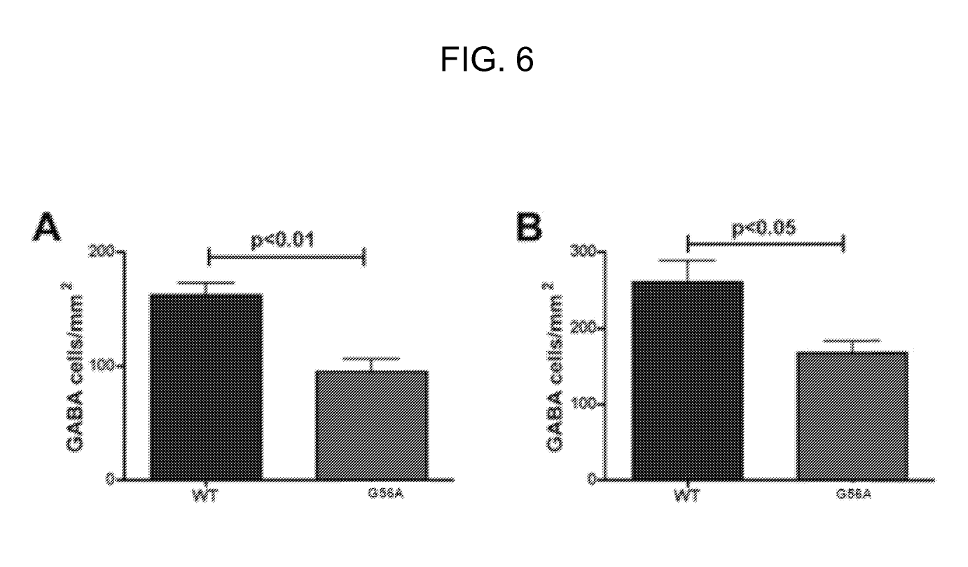

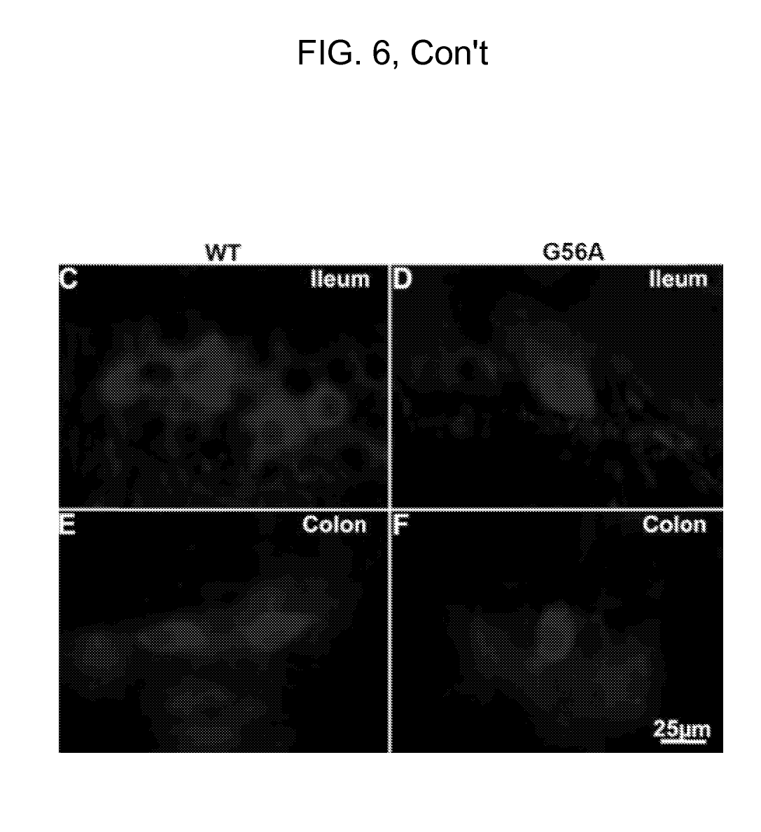

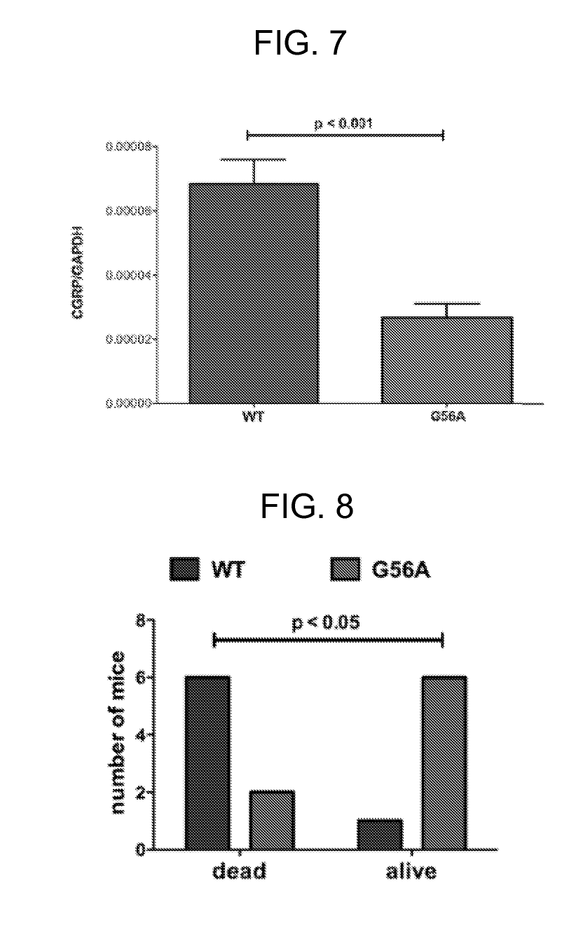

Total and late-born submucosal neurons were deficient in G56A mice (FIGS. 4A-4I), as were total and late-born myenteric neurons. (FIGS. 5A-5F and FIGS. 6A-6F). Accordingly, the abundance of transcripts encoding CGRP was low in the ilea of G56A mice compared to WT mice (FIG. 7).

Thus, the G56A mutation in SERT results in a slowing of intestinal motility, exhibited as an increase in total GI transit time, slow ejection of a bead placed into the rectum, slow small intestinal transit, and deficient frequency, velocity, and length of CMMCs in vitro. The latter indicates that the enteric nervous system (ENS) of the mouse is not functioning properly. Indeed, it is not because of the actions that serotonin normally exerts as a growth factor, which promote the development of those enteric neurons that are born later than serotonergic neurons during development. The total number of nerve cells in the mouse as well as the specific late-born neurons that are known to be regulated by serotonin (dopaminergic neurons, GABAergic neurons, CGRP-expressing neurons) are all deficient.

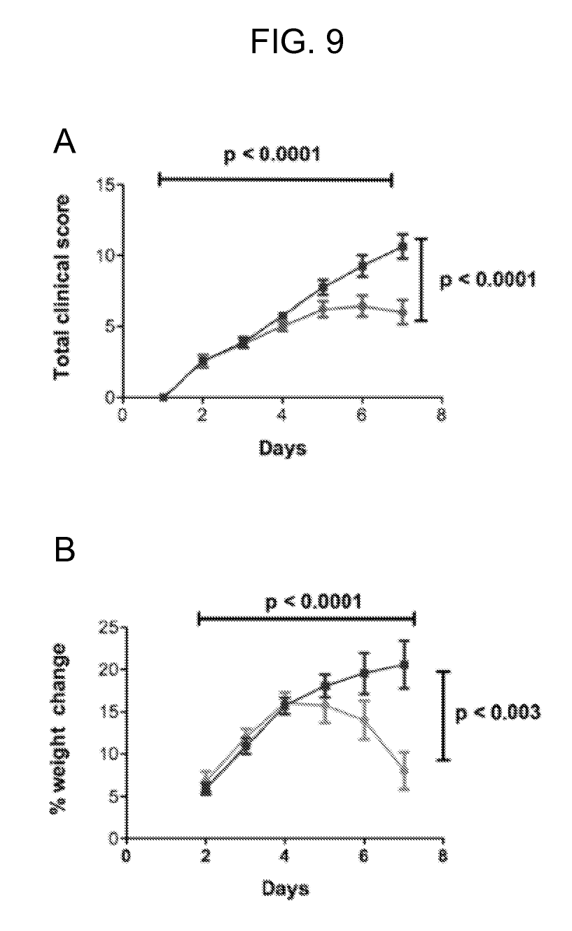

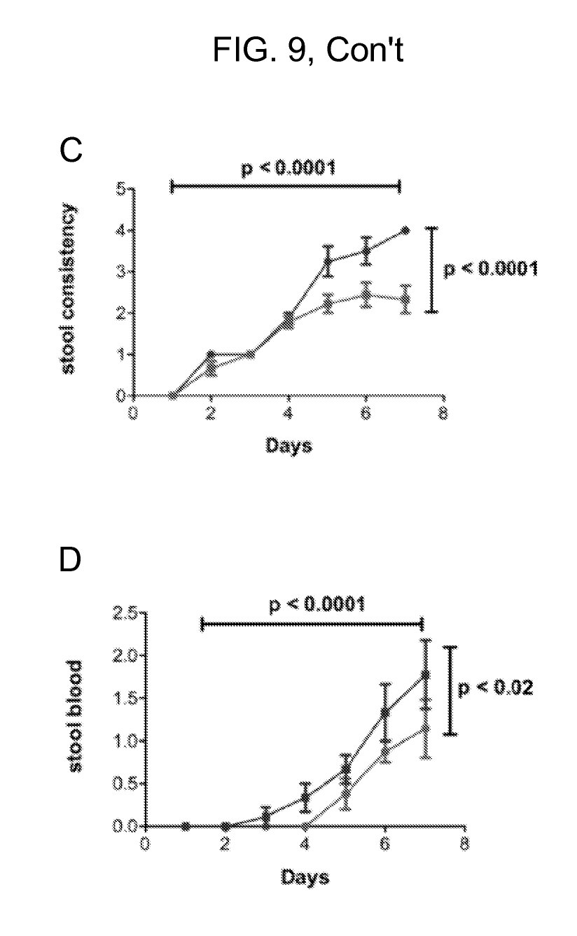

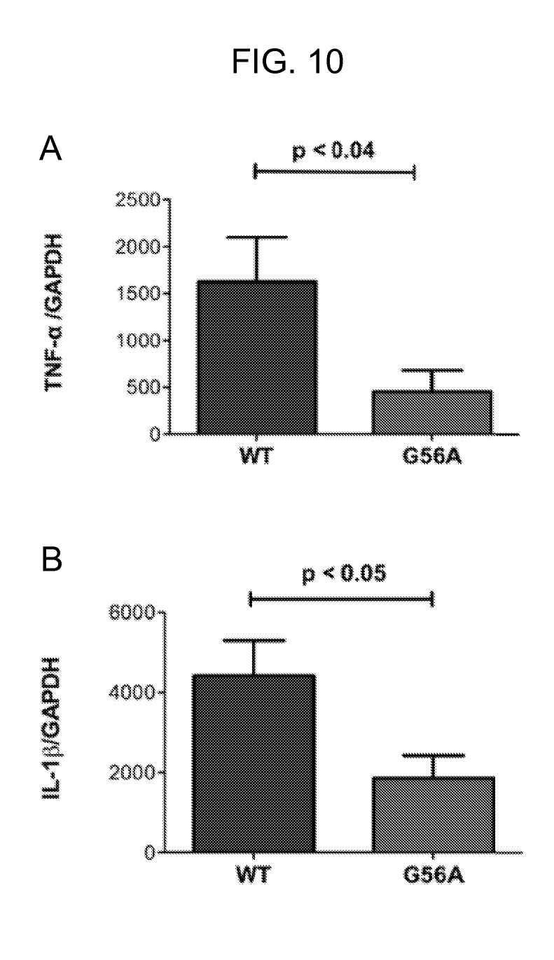

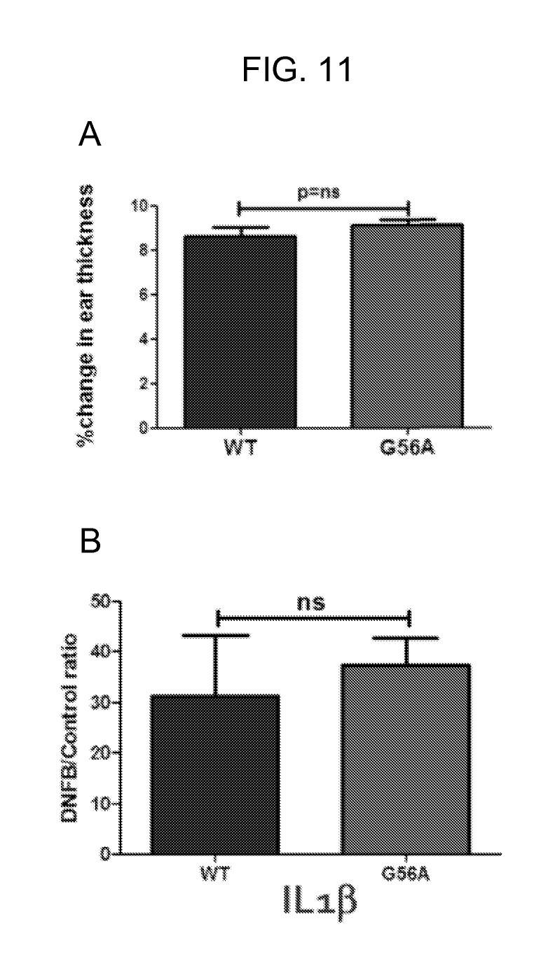

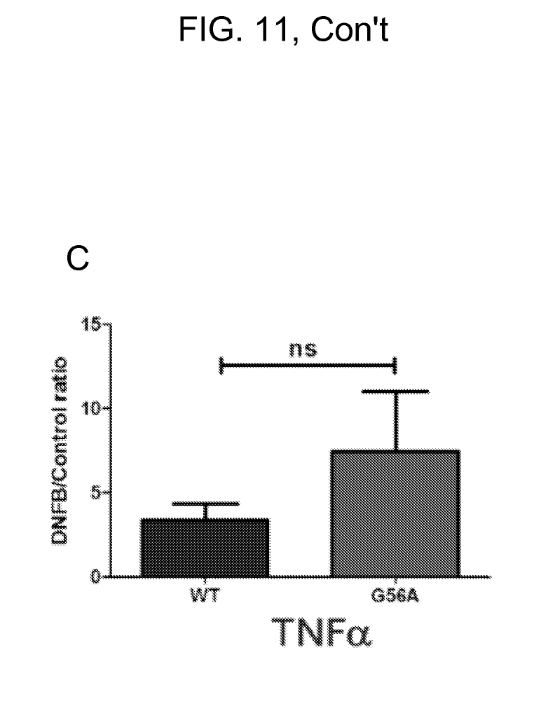

TNBS-induced colitis resulted in higher mortality rates in WT mice compared to G56A mice (FIG. 8). Total clinical score (FIG. 9A), percent weight change (FIG. 9B), stool consistency (FIG. 9C), and stood blood (FIG. 9D) were all higher in WT mice than in G56A mice. Moreover, TNF-α (FIG. 10A) and IL-1β (FIG. 10B) expression levels were higher in WT mice versus G56A mice. The extra-enteric immune response did not appear to be affected by the G56A mutation however. Ear thickness changes due to exposure to DNFB were not significantly different between WT and G56A mice (FIG. 11A), whereas IL-1β (FIG. 11B) and TNFα (FIG. 11C) transcript levels also did not exhibit significant differences.

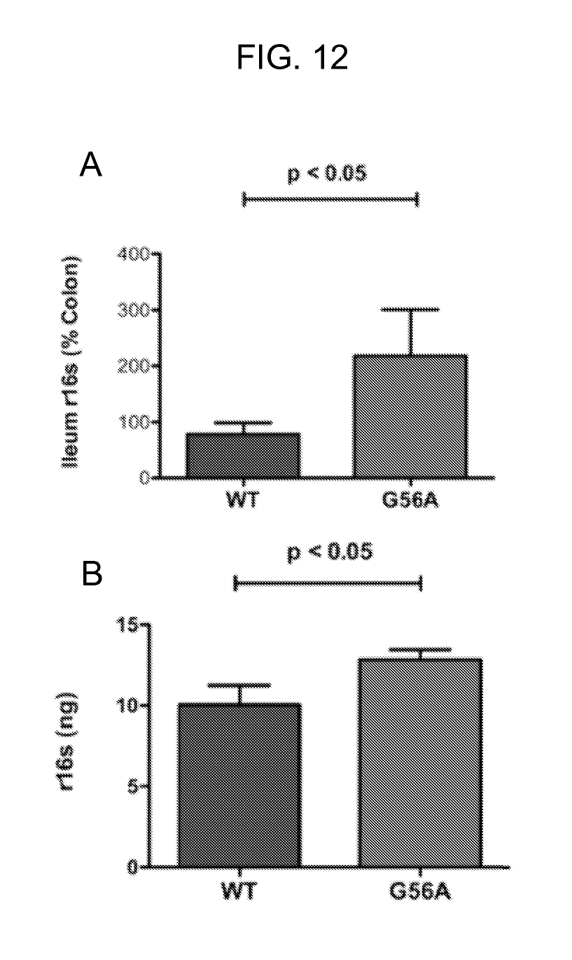

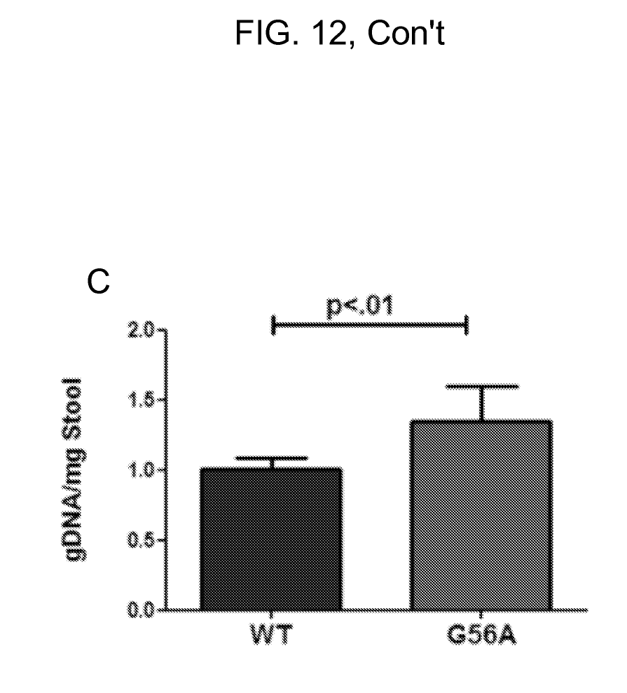

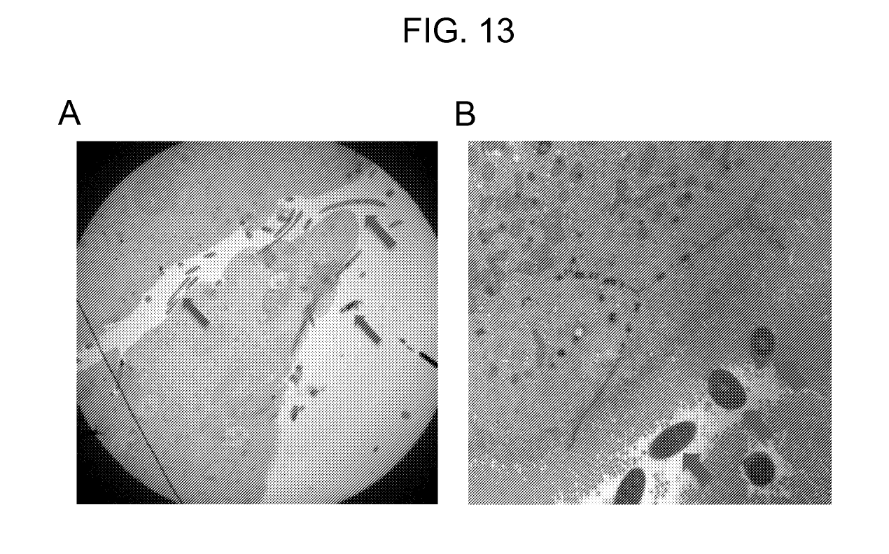

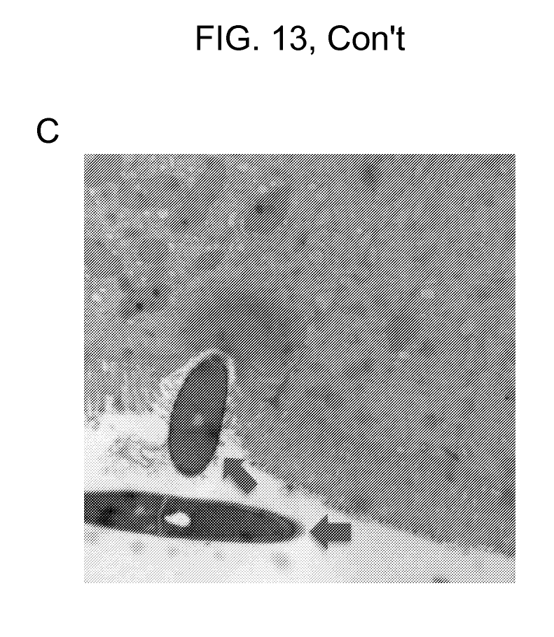

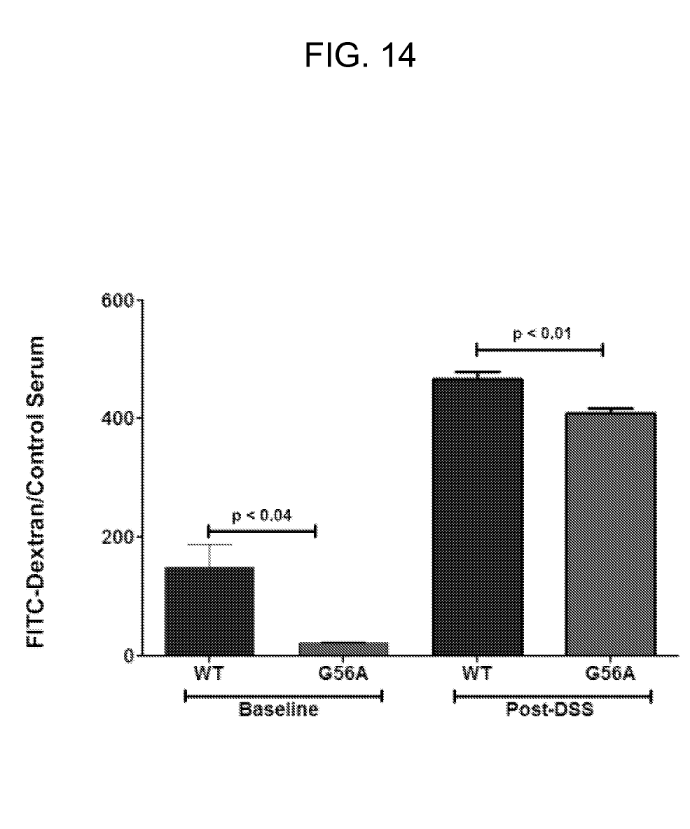

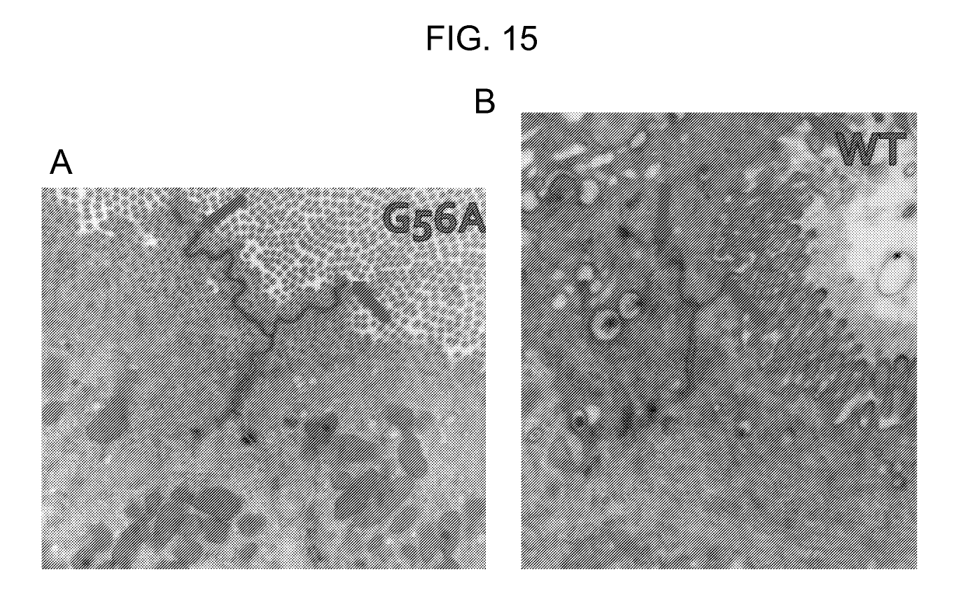

Bacterial load, SIBO, and invasion were greater in G56A versus WT mice as measured in the small intestine (FIG. 12A), mesenteric nodes (FIG. 12B), and stool (FIG. 12C). Ribosomal 16S RNA was used to quantify bacterial load. Indeed, more bacteria were seen in the small intestines of G56A mice than in those of WT mice. (FIGS. 13A-13C). Intestinal permeability was greater in WT mice (FIG. 14) while transepithelial transit of HRP was blocked at tight junctions in both WT and G56A mice. (FIGS. 15A-15B)

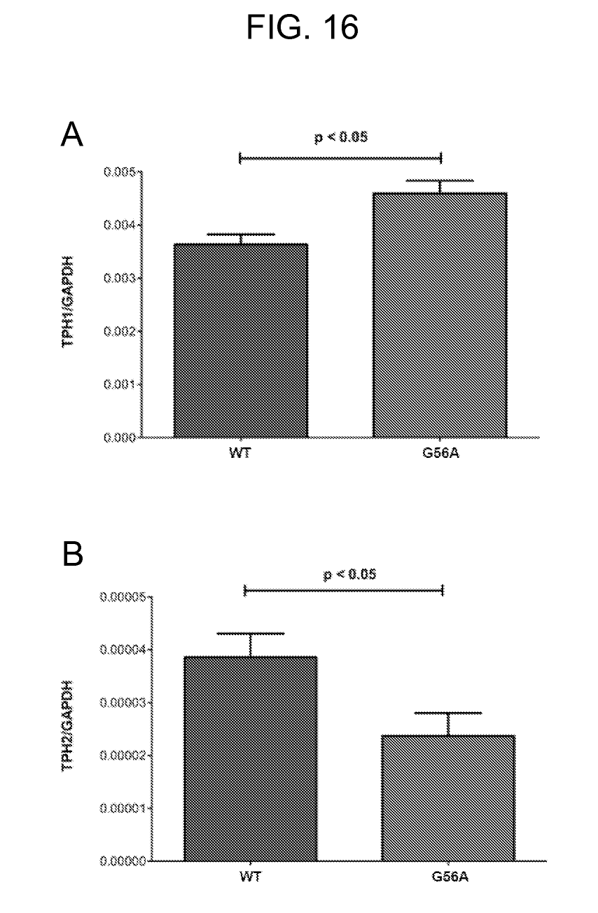

TPH1 expression was elevated in G56A mice compared to controls. (FIG. 16A). High TPH1 in G56A mice suggests that 5-HT biosynthesis increased to compensate for decreased 5-HT effects, explaining the hyperserotonemia observed in G56A mice. At the same time, TPH2 was decreased in G56A mice compared to WT mice (FIG. 16B). Low TPH2 suggests compensation, decreasing intraneuronal 5-HT biosynthesis to balance uptake and maintain a constant level of neuronal 5-HT.

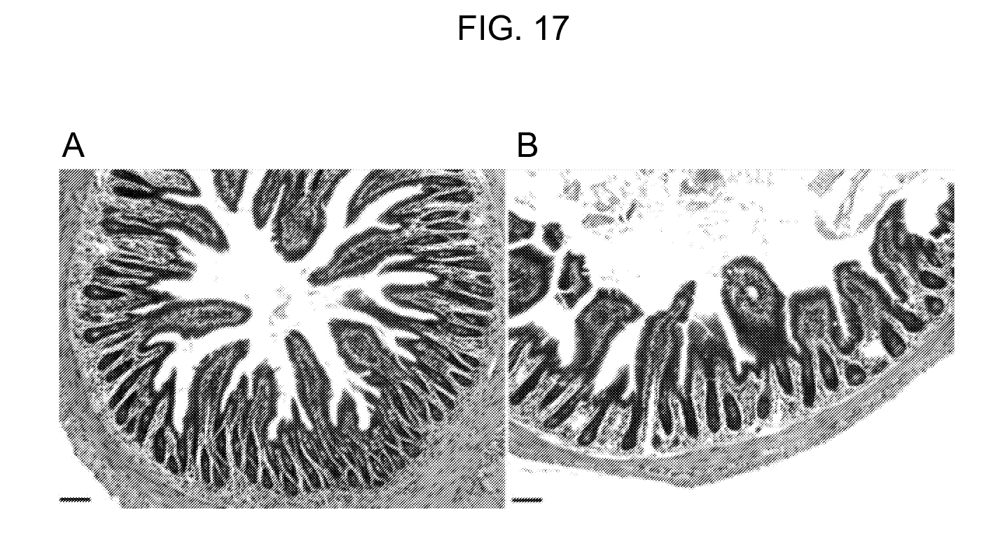

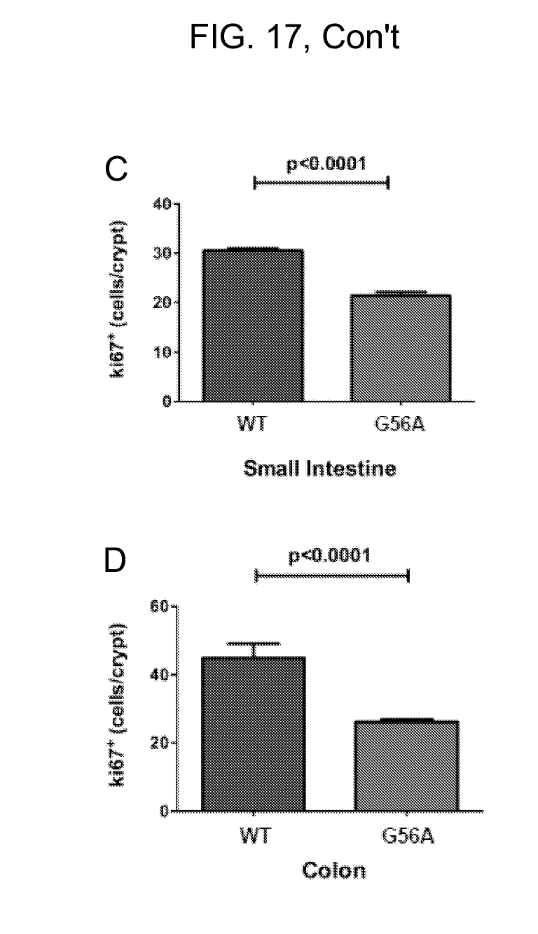

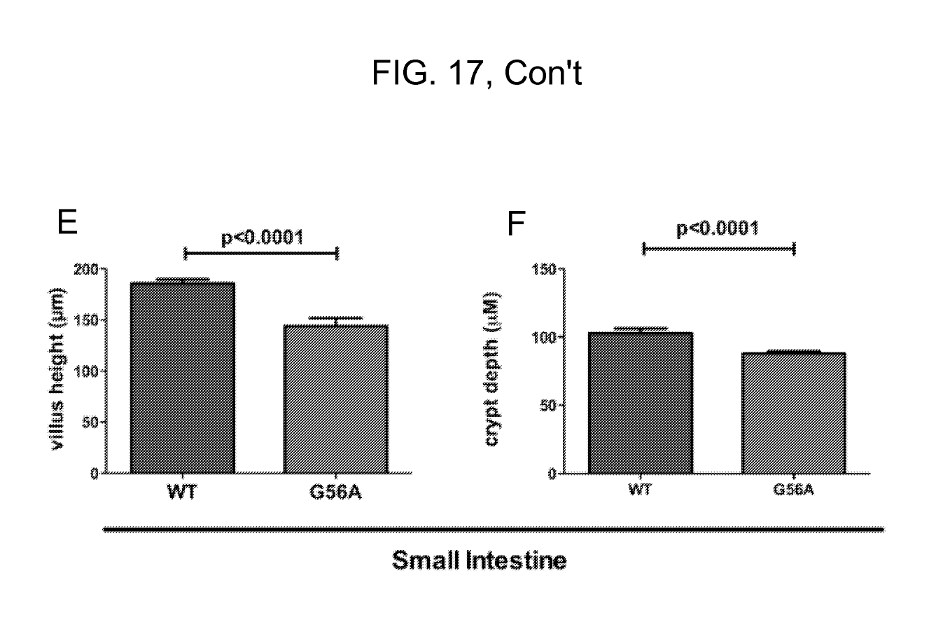

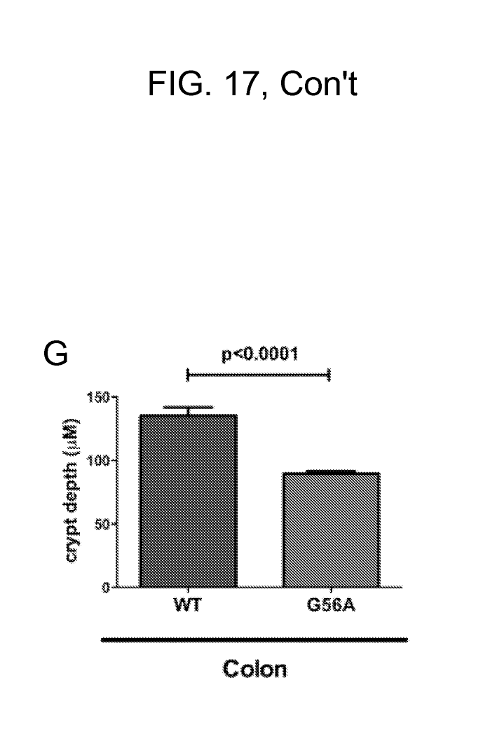

Villus height, crypt depth, and proliferation were all decreased in G56A mice (FIGS. 17A-17G).

In sum, the G56A mouse expresses a mutant form of SERT that is expressed in a subset of ASD patients. Mice that express G56A display ASD-like behavior and a GI phenotype consisting of slow GI transit, colonic motility, blunted 5-HT response, and abnormal CMMCs, as well as GI-specific resistance to TNBS-induced colitis, increased bacterial load, invasion, and SIBO, deficient mucosal maintenance indicating decreased epithelial proliferation, and increased expression of TPH1 alongside decreased expression of TPH2.

The G56A phenotype combines properties of TPH1 knockout (mucosal) and TPH2 knockout (neuronal) mice and supports the idea that 5-HT is a multifunctional enteric signaling molecule that functions abnormally in ASD.

### Example 4

### Prucalopride Rescues Gastrointestinal Motility in G56A SERT Mice

The ENS develops from neural crest-derived precursor cells (ENCDC). These ENCDC give rise to neurons that are born in a phenotype-related sequence. Because serotonergic neurons are among the first to terminally differentiate, they coexist with still dividing ENCDC. As a result, 5-HT from enteric serotonergic neurons can influence the differentiation of those neurons that follow the withdrawal of serotonergic neurons from the cell cycle. Indeed, the total number of enteric neurons and particularly dopaminergic, GABAergic, and CGRP-expressing neurons, all of which are late-born, are deficient in transgenic tryptophan hydroxylase 2 knockout (TPH2KO) mice, which cannot synthesize neuronal 5-HT, and in mice that carry a gain-of-function mutation, G56A, in SERT, the mutation causing 5-HT to be cleared from its receptors too rapidly.

5-HT₄ agonists, including prucalopride, stimulate enteric neurogenesis in wild-type adult mice but not in those lacking 5-HT₄ receptors. The hypothesis was tested that 5-HT-promoted enteric neurogenesis is 5-HT₄-mediated. The responsible receptor was also sought. ENCDC were isolated from E15 fetal mouse gut with antibodies to p75NTR and cultured in serum-free media. 5-HT and the selective 5-HT₄ agonists, prucalopride (2.5 μM) and BIMU-8 (2.5 μM), enhanced the development/survival of total enteric neurons; moreover, the effect of prucalopride/BIMU-8 on the development/survival of late-born neurons was significantly greater than on total. Prucalopride/BIMU-8 increased development/survival of the late-born enteric neurons that express TH or GABA. Neither prucalopride nor BIMU-8 enhanced development/survival of early-born (calretinin) neurons. The 5-HT₄ antagonist, GR113808 (1.0 μM), prevented 5-HT, prucalopride, or BIMU-8 from enhancing development/survival of total dopaminergic or GABAergic neurons. These observations suggest that 5-HT₄ stimulation is sufficient to account for the ability of 5-HT to promote enteric neurogenesis and that late-born enteric neurons are selectively 5-HT₄-sensitive.

The hypothesis, suggested by the results above, that treatment of developing mice with a 5-HT₄ agonist could rescue the ENS from the effects of a deficiency of neuronal 5-HT, was tested. It would be advantageous to rescue the G56A mouse by providing a small molecule to stimulate the type of serotonin receptor (thought to be the 5-HT₄ receptor) that would restore the motor defects to the bowel of G56A mice. In addition to their ENS defects, total GI transit time and colonic motility are significantly slower in G56A mice than in WT littermates, as discussed above.

Prucalopride was given orally to pregnant dams in early pregnancy, such as beginning at days E1-E12 (E1 is the day after a plug was discovered in the vagina, indicating that the mice had mated). The dose of prucalopride was 24 mg into 350 ml of water and the mice were assumed to drink about 5 ml/day. After birth, the prucalopride was continued in the drinking water, and was thus delivered to the newborn and suckling pups via breast milk. When the pups were weaned, the prucalopride was stopped; therefore, the exogenous 5-HT₄ stimulation was allowed to cease. GI motility was then tested in the offspring at ages 2-3 months. GI motility in the prucalopride-treated animals was found to have been restored to normal and was equivalent to that found in WT littermates. At this time, any improvement over the baseline properties of G56A mice had to have been due to rescue because the prucalopride had washed out of their system. Prucalopride-treatment of fetal and nursing WT mice was not found to exert significant effects on GI motility in the mature animals.

The results show that treatment of mice with the 5-HT₄ agonist prucalopride can rescue mice from the enteric development defects associated with a mutation in SERT that reduces the efficacy of enteric neuronal 5-HT as a growth factor. Because the G56A SERT mutation in humans has been linked to ASD, it is believed that the GI problems that are commonly found in ASD can be alleviated or prevented by administering a 5-HT₄ agonist, such as prucalopride, prenatally or to infants thought to be at risk of ASD.

### DOCUMENTS

KIMBALL, E. S., et al. (2005). Acute colitis induction by oil of mustard results in later development of an IBS-like accelerated upper GI transit in mice. American Journal of Physiology—Gastrointestinal and Liver Physiology 288(6): G1266-G1273.
LI, Z. S., et al. (2006). Physiological modulation of intestinal motility by enteric dopaminergic neurons and the D2 receptor: Analysis of dopamine receptor expression, location, development, and function in wild-type and knock-out mice. The Journal of Neuroscience 26(10): 2798-2807.
MARGOLIS, K. G., et al. (2011). Enteric neuronal density contributes to the severity of intestinal inflammation. Gastroenterology 141(2): 588-98.
MCELHANON, B. O., et al. (2014). Gastrointestinal symptoms in autism spectrum disorder: A meta-analysis. Pediatrics 133: 872-883.
MILLER, M. S., et al. (1981). Accurate measurement of intestinal transit in the rat. Journal of Pharmacological Methods 6(3): 211-217.
ROSE'MEYER, R. (2013). A review of the serotonin transporter and prenatal cortisol in the development of autism spectrum disorders. Molecular Autism 4(37): 1-16.
VEENSTRA-VANDERWEELE, J., et al. (2012). Autism gene variant causes hyperserotonemia, serotonin receptor hypersensitivity, social impairment and repetitive behavior. Proceedings of the National Academy of Sciences 109.14: 5469-5474.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating or ameliorating the effects of a disease associated with altered serotonin transporter molecule (SERT) activity in a subject in need thereof comprising administering to the subject an effective amount of a 5-HT₄ agonist or a pharmaceutically acceptable salt thereof.

2. The method according to claim 1, wherein the subject is a mammal.

3. The method according to claim 2, wherein the mammal is selected from the group consisting of humans, primates, farm animals, and domestic animals.

4. The method according to claim 2, wherein the mammal is a human.

5. The method according to claim 1, wherein the SERT activity is increased in the subject compared to a subject that does not have the disease.

6. The method according to claim 5, wherein the SERT activity is increased in the gastrointestinal tract of the subject with the disease.

7. The method according to claim 1, wherein the subject has a mutation in a gene encoding SERT.

8. The method according to claim 7, wherein the mutation is a G56A mutation in the gene.

9. The method according to claim 1, wherein the disease is selected from the group consisting of autism spectrum disorder, attention deficit hyperactivity disorder (ADHD), bipolar disorder, Tourette's syndrome, chronic intestinal pseudoobstruction (CIP), functional gastrointestinal disorders (FGID), irritable bowel syndrome (IBS), chronic constipation, functional dyspepsia, and combinations thereof.

10. The method according to claim 1, wherein the disease is autism spectrum disorder.

11. The method according to claim 1, wherein the 5-HT₄ agonist is selected from the group consisting of BIMU-8, cisapride, CJ-033,466, ML-10302, mosapride, prucalopride, renzapride, RS-67506, RS-67333, SL65.0155, tegaserod, zacopride, ATI-7505, velusetrag (TD-5108), levosulpiride, cinitapride, metoclopramide, pharmaceutically acceptable salts thereof, and combinations thereof.

12. The method according to claim 1, wherein the 5-HT₄ agonist is prucalopride or a pharmaceutically acceptable salt thereof.

13. A method for treating or ameliorating the effects of elevated serotonin transporter molecule (SERT) activity in the gastrointestinal tract of a subject in need thereof comprising administering to the subject an effective amount of a 5-HT₄ agonist or a pharmaceutically acceptable salt thereof.

14. The method according to claim 13, wherein the 5-HT₄ agonist is prucalopride or a pharmaceutically acceptable salt thereof.

15. The method according to claim 13, wherein the subject has autism spectrum disorder.

16. The method according to claim 13, wherein the subject is human.

17. A method for treating or ameliorating the effects of a gastrointestinal abnormality associated with autism spectrum disorder in a subject in need thereof comprising administering to the subject an effective amount of a 5-HT₄ agonist or a pharmaceutically acceptable salt thereof.

18. A pharmaceutical composition for treating or ameliorating the effects of a disease associated with altered serotonin transporter molecule (SERT) activity comprising (i) a pharmaceutically acceptable diluent or carrier and (ii) an effective amount of a 5-HT₄ agonist or a pharmaceutically acceptable salt thereof.

19. The pharmaceutical composition according to claim 18, wherein the 5-HT₄ agonist is prucalopride or a pharmaceutically acceptable salt thereof.

20. A kit for treating or ameliorating the effects of a disease associated with altered serotonin transporter molecule (SERT) activity comprising an effective amount of a 5-HT₄ agonist, packaged together with instructions for its use.

21. The kit according to claim 20, wherein the 5-HT₄ agonist is prucalopride or a pharmaceutically acceptable salt thereof.


SEQUENCE LISTING (28 sequences)

SEQ ID NO: 1 - DNA, 1364 bp, Homo sapiens
gaccctgtgggcattgaaatccaactcactcatgcttatttcctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaaggtacaccgttctgcacaggggacatcatcaggaactcgagaaactgcccatacacaatgacccagaatccctggaatcatgcttctgattgaggacatggctcacaacttagccattcattcgcattcatgtttgcatgaacaggtcaccctggcatcacttctgaacctcatcaccaccagtgaggcatcaggtagtaggggctgagagcccagaggaggtacatggaggacagtgttgg

SEQ ID NO: 2 - DNA, 3331 bp, Homo sapiens
agatgagcagcgctccggccagggagggcgcgtgctggccagcccggaacgggtgagatcgctccagcccgcctctgtgcccactcccctgtgcgccctgccggcagagcggcacccccgctgtccctggcgcccagtgggggcggcagcgggcagggcagcggggaccaccggggctgggggctgttgagcccgtggagtccggctcggttggggagaaggacgatgcgcggcgagcccaggtgatccggggctggggtcctgcgcccgcagcccctggcccgcgcctcacgctcgccggctccccagcgcgggggctggagcccgcacccgagggggcggcagggagggcgcaggcagcggctgggagcgcagcggcagcggcggcagcagaagctcggctcagcggctgggggtggccgctcgaatctgccagggcacctcgctccctcgcctctggcagcgggaccctgtgggcattgaaatccaactcactcatgcttatttcctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaagggatgcagtggagtgtggtggccagtgggagagtcagtgtcacccgccagcaacttctcctttggtggctgctcagcccagtgacacttaggcccctgggacaatgacccagaagacagccatgcctccgaaagagggccaggtcctaagctgctgcttgtgcgcgactgcacccggtattctcttcacctgaggctttccgtccgccagtgcaggaacccggtgctcgctgggcttttcctctgagattccagcaggtggcgctggagggagtcaggggacataatggcctccttgttcactttttatttcccaacactccctcttcccagattctctgcttttgccgcgtggtctctggtgtctctgacatgttcccgtcactcactgtgtctgatctgtctcactcacgttctaagcacgatagcttgctgcgtcctatctcggtcactgatgtctccaaatacgccttctttgctgtgtgggcttccagatatcaacaaaatactccctctctgttcctgtcttagccagaggcgtcgctcctgtcttgcttacttgcttggtcacgtccgtcacagccatgttcaagctatcccctgcctcttcttcgccctgttctgtgccagacgctaacacacttttctccctgtgtctgggtccaaacgtgcatcctcttggttggcccccttgtcagtcccagacgtccacttgctcttccttatgtccaatgtctgtcctcctcttcaggacatgctccatttattttccatggaagacacagggtcagagatctgtcagaggaaaatggtctccacagcaactctggagggaagctgaagacagatagcttcctcctggtgtcctggccgctggcgtgtctgccctgagatgctcgctctcctgctgggctagaacgggacgtcttctaaaaagccttgggcagggctagcaacatcgtaggtgtttattaggcttcaggatctttcccatgaacttgtgatatcattttgaaacccttcaatctggctttgttgctgccaaaggagataactgttggaagttcacactggggaaactcattttcccctgcagctcccccaaggtgcaggagggcaggatccagcagcactggggatgccgaggcaatgctgtacttgaagccagaggacccaaattgccgccctggctgtgctgcaaacaagctgtgagatcttgaatagtgttctttcatctctggaccccactttccacatctgtgcactcaagcttggactagaggacctttaagttttctttctgtttctaagacaacagtatgctgctatagccgtctgctacctcgagaagccatggcctgcctgccttttggctttgtttcaaaggaacgaccctcctcatttgggccttttagacccctgacctttccagctttgtccttcagattaatctggatgtcttttattcaaagctgagtcagtggctcttgcgaatgccccatagcagaagtcccgggaaggtaaatgccaaacccagagccatgtgagattctgactttgatcttggctgtggtttaatagcatctcaggattatttatcatattctctttttttcttattgcttataaggtgtcagttattgtttccattgttgtttactttagttctggaactgaaaccgacaggaagaaactttggaataaggaagagaagattgaccaaaccatccaaatgccaaaaag

SEQ ID NO: 3 - DNA, 1283 bp, Homo sapiens
gaccctgtgggcattgaaatccaactcactcatgcttatttcctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaagttctggaactgaaaccgacaggaagaaactttggaataaggaagagaagattgaccaaaccatccaaatgccaaaaagaaaaagaaaaaaaaaagcctctctgtcctatgaagacttaattctgttggggagaaagtcatgttttagggaaggaaaatag

SEQ ID NO: 4 - DNA, 1238 bp, Homo sapiens
gaccctgtgggcattgaaatccaactcactcatgcttatttcctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaagattttgagctccttgaggactgtggccaattcttattgctcatttttttttcttagtgcccaacacaggtttttgtacactgaagttatcaaataaattcattggatgtaaaaaa

SEQ ID NO: 5 - DNA, 1440 bp, Homo sapiens
gaccctgtgggcattgaaatccaactcactcatgcttatttcctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaagtggctgttcccctgtctccagcttcctcctgctcttctgcaatagaccagttcctgtctaactctgaaagtgctctgtacaccgttctgcacaggggacatcatcaggaactcgagaaactgcccatacacaatgacccagaatccctggaatcatgcttctgattgaggacatggctcacaacttagccattcattcgcattcatgtttgcatgaacaggtcaccctggcatcacttctgaacctcatcaccaccagtgaggcatcaggtagtaggggctgagagcccagaggaggtacatggaggacagtgttgg

SEQ ID NO: 6 - DNA, 1440 bp, Homo sapiens; identical to SEQ ID NO: 5

SEQ ID NO: 7 - DNA, 4657 bp, Homo sapiens
agtggcggggtgcgctactcagcgcagcagcagcggcagaagcagggttctgcgctaaaggtggccgcagcaatctgctagggtgccgctctccctagccttccgcagccgcgtggtgctgggagtgtgccaggagatcagaaccccaggagcccacgtgtgcagggtcctgtgggcactgaaatccaacgtcctcatgcccatttcctgtaatggacaaacttgatgctaatgtgagttccaacgagggtttcaggtccgtggagaaggtcgtgctgctcacgttccttgcagtggttatcctgatggccatcttgggcaacctgctggtgatggtggctgtgtgcagggacaggcagctcaggaaaataaaaaccaactatttcattgtgtctctcgcctttgctgacctgctggtttcggtgctggtgatgccctttggtgccattgagctggtccaagacatctgggcttatggggagatgttctgcctggtccggacctctctggatgtcctacttaccacagcatcgatctttcacctgtgctgtatttccctggacaggtattacgccatctgctgccagcctttggtttataggaacaagatgacccctctacgcatcgcattaatgttgggaggctgctgggtccttcccatgtttatatcttttctccccataatgcaaggctggaacaacatcggcatagttgatgtgatagagaaaaggaaattcagccacaactctaactccacgtggtgtgtcttcatggtcaacaagccctatgctatcacctgctctgtggtggccttctacatcccgtttctcctcatggtgctggcctattaccgaatctatgtcactgctaaggagcatgcccagcagatacagatgttacaacgggcaggagccacctctgaaagcaggccccagccagctgaccagcacagcacacatcgcatgaggacagagaccaaggcagccaagactttatgtgtcatcatgggctgcttctgtttctgctgggcccccttctttgtcaccaatattgtggaccctttcatagactacactgtccccgagcaggtgtggactgctttcctctggcttggctatatcaattcggggttgaacccttttctctatgccttcttgaataagtctttcagacgtgccttcctcatcatcctctgctgtgatgatgagcgctacaaaagaccccccattctgggccagactgtcccctgttcaaccacaaccattaatggatccacccatgtactaagggatacagtggaatgtggtggccagtgggagagtcggtgtcacctcacagcaacttctcctttggtggctgctcagcccagtgatacttaagcccctgggacaatgacctagaagacagctgtggcttgaaacgaagccagtcctaagctgctacttgtgtgtatgtggctggccctggcactctcttctccaaggctttccaagagcatgaggcaatccgcactggactttcccgccaggaatccagcaggcggtatttgaggaagtcaggggagagaagggcttcctccttcccttcgctttctgtttctcaagagttcttcttcctggagtctccactcttgcttggtggtctctgaagtctatgacctagtctccttgctgttttcagtctgtcctgtaaacgtttactgtgttcaattttctgtttccaaacatgccttctttgaagtgtcatcttacagatactgtcaaaacatgtgcatgtctttatcacatttcttgcttggtcacatctaccatagctgtgttcagacactccaccatctttcctttgctctcttctctgctgggcccttgttcactttctttcctttgtctgggtatagacatgtatcctcttggccaaccctgtgtcagccacagaactctgttttctttttcctgtgtttagtggtaccattctcttcaggacaggctccatccatttgcagtcggagacacagaagagagatccgtcagtgggaaacggtctctgcagaaaccctggaaggaagtcaggaccagatctatctcatctgagagatttcactttcctttcagcttagcacacctaaagagccttaggcagggctggttcatcacatgcatctattaagagatgctcagactcttctatgagcttgtggtgaaacacacagtagcagctcaaaacctcagagtatggctttgctgcttccagtggaccttactgatggatgctcaccttgtggagccttgtcactcttgctatcttctaggtggagaagtccagaaatgctagtaatgagaaaacagaatctggctccaagtcagaggacccaaatgtaagcccaggccctgcttctgaaacgctgtgagaccatgaacaatgtctttccatctatgaatttcactctctacattgccaaatttaacttggagcagaggagtctttaagatttctttccatttctaagacaataggatgctgctatagcctgccatctgctacctcaaaagctcatggcctgcctacctgttggctttgtttcaaaggaatggccctcctgatttgggcatgctagacttctgacttgatcatctgtgcaaactggtgaaacacagtgaacctggtgtctttcactcaaagccgagtccatagtttgtctcaactacccgagagcaaaggctctggtgatggaagcaccacttctcagagccaggtgaggttcagctttcgccttgactgtggtttgattccacctcaggactatgcactgcaccgttctcttgagcttctcgtggaatggcacttgctattcctgtcagcattgactttcattctggaactgaacttggcaggaaatagactttggaatgaggaagagaagattgccaaaaccagtcaaatgccaaaaaagaattctctgttatgtgaagacttaaattttcttgagcacgagtcttagcaaaaggaagtagaccctgagagagaactcctatatgctgctggggaaagaaaaaaaaaagctctgtgctaagaaatattgatgctgagatggttcgtgtcaaaactgatccaatggagcagttctcactggaggcctcttctcgtcaaccatgaagagagttgaagcatcaccttgcatactgacgttgtagcgggatcagtccactttgatttttaactccttgcttccttgccgtggctaagctcagcactcatttggaggcaagcagtgttttgaagctggttctttttaatacaagagatgagatgcaaccaacttgcctcgactgaccatatgcccaccccgtcaacgaacatggtggtgggtgagtggctcaggcactgagtcaatgatggactggctgagtggatggaattgaaagctggatggtgtgagcctcaggaacacttctgagtactctgaaacagttgatgtggagagaaattatgtttatggagacattttgtaaaatataaaggggcagctcatatgtttgttattattatgggtattttgaattactgtgctctcaagaccacttttttttttcactaaaaaatgaggtcagtttagagtgaggtttctgtgttggaaacaaaacccagagcaacccttgggtaccaacatgcccctgggtactcattccttattaattgcttctcattggttgcttagcccacacatttatacatcacccttcatttaaagggatttctttgttctcaaaggggtgaatagagaaggagaccgtgggcaagttttggtaagaatggctctaattttatatgtaacttcatagtttcataagctaagttcatatacctgctttttatataaaaatgtatctgaaaaaaattccctatacataaaactaattcccaccttacaaataaaatatatacataatatatatttatttaatatatatacatatacactatatatgtacatatataatatatattatattaaatgtggaccaatgtatgaatggccagctgacaagaatgttacataatgggtcactaagttactgaacaaggatagactttgatatttcctgacctgatgataagccactgttgactacagcataatgtcgttttatctaatacttttattttgtttacagtttgtccatgtgtagttaccagtggggccacttgtattatttgtagttcaatgaagcctctagtataatcaatccagtaagtacatgtgtgcctgctgttgagaacatgaagcataaatcttacgagtttaggggttggggagttccaactatggtgtcatgaactggttggctatacctgtggtttattgacgctaatatttttaaggtaccaagagccctgattattaccttgtttccatggaaagcatccttggttttatgacagatccttttgatgcagtttccaggaatatgttcttggaaaacacagggaccttcacaggttttgtttttaaactgattaattatatttattgataaaatattgttatattacacatgtttgtatgacttcatctgtacatgtttaaataagttatacattatgacaaatcttaaatcataaaagaaataattgagttttgagtatgcaataaaatgcc

SEQ ID NO: 8 - protein, 387 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRYTVLHRGHHQELEKLPIHNDPESLESCF

SEQ ID NO: 9 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 10 - protein, 428 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRTDFLFDRDILARYWTKPARAGPFSGTLSIRCLTARKPVLGDAVECGGQWESQCHPPATSPLVAAQPSDT

SEQ ID NO: 11 - protein, 428 aa, Homo sapiens; identical to SEQ ID NO: 10

SEQ ID NO: 12 - protein, 428 aa, Homo sapiens; identical to SEQ ID NO: 10

SEQ ID NO: 13 - protein, 428 aa, Homo sapiens; identical to SEQ ID NO: 10

SEQ ID NO: 14 - protein, 388 aa, Homo sapiens
MDKLDANVSSNEGFRSVEKVVLLTFLAVVILMAILGNLLVMVAVCRDRQLRKIKTNYFIVSLAFADLLVSVLVMPFGAIELVQDIWAYGEMFCLVRTSLDVLLTTASIFHLCCISLDRYYAICCQPLVYRNKMTPLRIALMLGGCWVLPMFISFLPIMQGWNNIGIVDVIEKRKFSHNSNSTWCVFMVNKPYAITCSVVAFYIPFLLMVLAYYRIYVTAKEHAQQIQMLQRAGATSESRPQPADQHSTHRMRTETKAAKTLCVIMGCFCFCWAPFFVTNIVDPFIDYTVPEQVWTAFLWLGYINSGLNPFLYAFLNKSFRRAFLIILCCDDERYKRPPILGQTVPCSTTTINGSTHVLRDTVECGGQWESRCHLTATSPLVAAQPSDT

SEQ ID NO: 15 - DNA, 6615 bp, Homo sapiens
gctcctccctgcgagcgtgtgtgtgtgtcgggggtccctcccctcctggctctggggtcgggcgcgcaccccgccccgtagcgcggcccctccctggcgagcgcaaccccatccagcgggagcgcggagccgcggccgcggggaagcattaagtttattcgcctcaaagtgacgcaaaaattcttcaagagctctttggcggcggctatctagagatcagaccatgtgagggcccgcgggtacaaatacggccgcgccggcgcccctccgcacagccagcgccgccgggtgcctcgagggcgcgaggccagcccgcctgcccagcccgggaccagcctccccgcgcagcctggcaggtctcctggaggcaaggcgaccttgcttgccctctcttgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaagcttgtgagtctgtgtatattgttgtttcagtgtattcttatctctagtccaatattttgggcccattacaaatatatgaattccccaaatttttcttacattaacaaattctaccaactcaattgtgtatggaggttattatttgaagggtacaatcactacaacatgctctgccacccactccttttccagtgacactacttgagccacacactttcctttacaggccagcctctggcgtttgctgcacctcattgccaccttcctgtctctctgtgctaaacattcaggacagtgttccacaggcagatctggcctatttcattagtcaccatggcttggctgtgaagtacgttgaaggtggatcttgtcacatgccccttcagtgttcacctggccctctggtttaagttctgtctgccttacgtgactgagtttgactgtccaggttgctttgctcggtgaagagaggagggtaaatcggattctcgtttagcactgggttatacagatctggcaccctaacctaaaccaaggcatcttcactccaagagcagttggagagtctgggttagccttacgtggacctcgccgctcgctggcggtcacgattgtgagccctccagataatttttaaggttgagtctaagtaaggctgcttgggaaatggtcagctaagtaaatcacctttcatttcacataaggcccttaatatagataagtaaatttggcctttggtgtctcgtgactctcagaggcgtaggtagaggagcaaattaatatttgcagcatgggaattccttatcagaattttgaggggaataaatcctcatcagagacaaaaggacttaatcatctggccacctatcacttcagttctctgtataaatgaaatttaattctaacaaccttataaaaagaaggtccagacagcagaggaaacatcctgtccaattctaggttttcctcccttggcctcctttccccagcattgtctaccctggcccacttcctgcattctccccatgccctgctatttctgattctttgcttctcctagcgagatactttccttatatgatagctgctgagaagtttcccagaactgctagaggaaaagaagtggggaatttaggaaatatccctcactgacctaactccattatcttcactctttccttcttcctgccacctcatgcccattctctttactgtctagcatgctgaaagaaggaagtgatctaaatgccagcgtgttcagtggtaaatattagttggtgcaaaagaaaaaccatgattacttttgcactaacctaatagctttgcaaattttaagaacttgctttatgaagatattcggatatggattctccccaccccacatacttagacattgttcaaatatactacttttaaaaaaacaccttttcaaacagaattagcgttttgccaagtctggtattaatggaattgtacaggagctttgaaagttttcaaactttattaaactaaaaaaaaaaaatcgaaaatctctgtctgttccgcatagtatgcatttatttgacccctatttatcaatactatgatggggttttttttttttaaagaaaatttaagagtaggtaggtggattttaaaataatattttaaagaccttttatatctatatgtagcatttatagaaaaataaaaactaaaaatagaattgaattgtaacattatttaaggactgaagttttttttcttgtatcagtaagaaatacccaagaggctgggtgcagtgactcacacctgtaatcccagcactttgggaggctgcagtgggaggatcacatgacatcaggagtttgagaccagcttggccaacatagtgaaacgccgtctctattaaaaatacagaaaattagctgaatgtggtggcaggcgcctataattcctgctacttgggaggctgaggcaggagaattgcttgaacccaggaggcagaggttgcagtgagccaaacgttccactgcattccagcctggatgacaagagcgaaactccgtctcaaaaaaaaaaaaaaaaattgttatgcttagttccatggaaagactattctgaagctttaagtcttctttttctattttccatagtattgccccttccccacttcattgcttaactgtctctaaattttaatgataataatattttaaaggtcagataatgccattagaggcagagacaaacctggggaccaagctaatttctctgttattgagagcgctaaagacaggtgaactggagttttatttcctctgctagagtgaaataataccctctccaccaggcacaatggctcacacctgtaattccagcactttgggaggccaaggtgggcggatcacttgaggtcaggagttcgagaccagcctggccaacatggtgaaaccccatctctactaaaaatacaaaaattagccagccatggtggcatgctcctgtaatcccagctacttgggagactgaggtgggataatcacttgaacctgggaggcggaggttgcagtgagctgagattgtgccactgcactccagcctgggtgacagagcaagactccatctaaaaaacaaaaacaaaaacaaaaaaaccctctcaattggtttaataccacgatagaaaagataaatattttaggatggaatcttaaatatgtctgtccttttgtttcatatagttgaaatcaattcagtattgtttctacattaggtgtttcaaaaacagtcacctttgcaacagaagagctcttttgttgaaaatgattcacaatatatttcagttggaatgtcaggtggtatttctcttaccaacacctcacagaatctatgccaagctgctctaccaccaatgcaagtatttttttaaagctactaaaataaactttcttcaccagccaactctaatttggagacagtgtgcattggtgaaggacctcgtcagaataagtttgaatgtctactgaactaagaagaattttgtcgtttgggggagagaatagatggcatcagtccttcaattctgtaactgaagactccaattatagtagataagaattgtgtctagcaatttttaaacactgacagtccaaacaaaaatatttggtgaggaaggatgtcccatatttttgcttaaatatcataaacaaatatgagcatttactaattttttaaatggcattttgaaagattattcttatttacactctaaaattaaaggtgtactttatcttaagaaaatgatatattaaaaattcatattttaaaagataaaattggggaatttacagttattttgtgaatggcctttaaactatgatttgatctatatacaacttttcagaatacttttgattgtgtgttggacatatctaaaattaattttatctggcagaattaaacctaaatttatttgtataaaagagtcccctttctaaaattcatacagtgcccttgtattcatttatgcagtgtttataaatatttcaagttagattgtgagatattaataaatgatttatgctgtgaaaaaaaaaaa

SEQ ID NO: 16 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 17 - DNA, 2747 bp, Mus musculus
agcggccgcgccggtgcctcgagggcgcgagggtcgagccgcctccgcagcccgggacccgccgccgcgcctcccgcagagctctcagtcttgtctccataacactgagaggagattcaaaaccaagaaccaagagctagtcagggtccttggcagatgggcatccgcaccactgactgaccagcagcatggagaccacacctttgaattctcagaaagtgctgtcagagtgtaaggacaaagaggactgccaagaaaatggtgttctgcagaagggtgtccccacaccagcagacaaggcagggcctggacaaatatccaatgggtactccgcagttcccagtacaagcgctggggatgaagcgccacactctacgccagctgccaccaccaccctggtggctgagattcaccaaggggaacgggagacctggggcaagaagatggatttcctcctgtctgtcattggctatgccgtggacctgggcaacatctggcgttttccctacatatgctaccagaatggtggaggggccttcctcctcccttacaccatcatggccatctttggggggatcccactcttctacatggagctcgccctgggccagtaccaccgaaatgggtgcatttctatatggaagaagatctgcccgattttcaaaggcattggctatgccatctgcatcattgccttttatatcgcctcctactataacaccatcatagcctgggcgctctactacctcatctcctccttcacggaccagctgccctggaccagctgcaagaactcttggaacactggcaactgcaccaactacttcgcccaggacaacatcacctggacactccattccacgtcacctgctgaggagttttacttgcgccatgtcctgcagatccatcagtcaaagggactccaggacctggggaccatcagctggcagctggctctctgcatcatgctcatcttcaccattatctacttcagcatctggaaaggagtcaaaacgtctggcaaggtggtgtgggtgacagccaccttcccttacattgtcctttctgtcctgctggtgaggggagccacccttcctggagcctggagaggggttgtcttttacttgaaacccaactggcagaaactcttggagacaggggtgtgggttgatgctgcggctcagatctttttctctcttggcccggggtttggggttctcctggcgtttgctagctacaacaagttcaacaacaactgttaccaagatgccctggtgaccagtgtggtgaactgcatgacgagcttcgtctctggctttgtcatcttcacggtgcttggctacatggctgagatgaggaacgaagacgtgtccgaggtggccaaagacgcgggccccagcctccttttcatcacatatgcggaggcaatagctaacatgccagcatccacattctttgccatcatcttcttcctcatgttaatcacgctgggtttggatagtacgttcgcaggcctggaaggtgtgatcacagctgtgttggatgagtttcctcacatctgggccaagcgcagggaatggtttgtgctcatcgtggtcatcacttgcatcttgggatccctgctcacactgacatcaggaggggcgtatgtggtgaccctgctggaggagtacgccacggggccagcagtgctcaccgtggctctcatcgaggccgtcgtcgtgtcttggttctatggaatcactcagttctgcagcgacgtgaaggaaatgctgggcttcagccccggatggttttggaggatctgctgggtggccatcagccctctgtttctcctgttcatcatttgcagttttttgatgagtccaccccaactccggcttttccaatacaattatccccactggagtatcatcttgggctactgcataggaacatcgtctgtcatctgcatccctatatacatcatttatcggctgatcagcactccagggacacttaaggagcgcattattaaaagtatcactcctgaaacaccaacggaaattccgtgtggggacatccgcatgaatgctgtgtaacacactgagggagaggacatggcctcccagcccccgactcctcatctctgaaaagccccaccttgactcctcccctctaagccaagctgatgatgtaaggtctttctccatggagtcacagtcctaaagactatggtgcccagactcttgtgggttccaaccacttctttccatgaactctcttggacttactgccacattagctggtgacacggctgagctgacttggatatgtgaggagaggaaggaggggatgaacgccacccagtcatcagctagcttcaggtttagattaggtctgtgaacgtctgtatcatgttctgggtatgatcatattgccctgcatctgtttgcttctaaagccttcagtgttcatgaatacataaaccacctaagagaaaacagggatgtcttgctagccatatatattttctcagtagcatagaactctgtagccggaatctactagaaccctgtaacccacgtgctgctgtgaggttaagaaaggaagacgtaaagacgctacactgaaaactgatatatatgtgtgagctcttgtgtctgtccattgttgtctgtgtcccctcaattccaacactccgggcccattacaaactatataaatggcctctaatttttcttacattaaacagattctacctactc

SEQ ID NO: 18 - protein, 2747 aa, Mus musculus
AGCGGCCGCGCCGGTGCCTCGAGGGCGCGAGGGTCGAGCCGCCTCCGCAGCCCGGGACCCGCCGCCGCGCCTCCCGCAGAGCTCTCAGTCTTGTCTCCATAACACTGAGAGGAGATTCAAAACCAAGAACCAAGAGCTAGTCAGGGTCCTTGGCAGATGGGCATCCGCACCACTGACTGACCAGCAGCATGGAGACCACACCTTTGAATTCTCAGAAAGTGCTGTCAGAGTGTAAGGACAAAGAGGACTGCCAAGAAAATGGTGTTCTGCAGAAGGGTGTCCCCACACCAGCAGACAAGGCAGGGCCTGGACAAATATCCAATGGGTACTCCGCAGTTCCCAGTACAAGCGCTGGGGATGAAGCGCCACACTCTACGCCAGCTGCCACCACCACCCTGGTGGCTGAGATTCACCAAGGGGAACGGGAGACCTGGGGCAAGAAGATGGATTTCCTCCTGTCTGTCATTGGCTATGCCGTGGACCTGGGCAACATCTGGCGTTTTCCCTACATATGCTACCAGAATGGTGGAGGGGCCTTCCTCCTCCCTTACACCATCATGGCCATCTTTGGGGGGATCCCACTCTTCTACATGGAGCTCGCCCTGGGCCAGTACCACCGAAATGGGTGCATTTCTATATGGAAGAAGATCTGCCCGATTTTCAAAGGCATTGGCTATGCCATCTGCATCATTGCCTTTTATATCGCCTCCTACTATAACACCATCATAGCCTGGGCGCTCTACTACCTCATCTCCTCCTTCACGGACCAGCTGCCCTGGACCAGCTGCAAGAACTCTTGGAACACTGGCAACTGCACCAACTACTTCGCCCAGGACAACATCACCTGGACACTCCATTCCACGTCACCTGCTGAGGAGTTTTACTTGCGCCATGTCCTGCAGATCCATCAGTCAAAGGGACTCCAGGACCTGGGGACCATCAGCTGGCAGCTGGCTCTCTGCATCATGCTCATCTTCACCATTATCTACTTCAGCATCTGGAAAGGAGTCAAAACGTCTGGCAAGGTGGTGTGGGTGACAGCCACCTTCCCTTACATTGTCCTTTCTGTCCTGCTGGTGAGGGGAGCCACCCTTCCTGGAGCCTGGAGAGGGGTTGTCTTTTACTTGAAACCCAACTGGCAGAAACTCTTGGAGACAGGGGTGTGGGTTGATGCTGCGGCTCAGATCTTTTTCTCTCTTGGCCCGGGGTTTGGGGTTCTCCTGGCGTTTGCTAGCTACAACAAGTTCAACAACAACTGTTACCAAGATGCCCTGGTGACCAGTGTGGTGAACTGCATGACGAGCTTCGTCTCTGGCTTTGTCATCTTCACGGTGCTTGGCTACATGGCTGAGATGAGGAACGAAGACGTGTCCGAGGTGGCCAAAGACGCGGGCCCCAGCCTCCTTTTCATCACATATGCGGAGGCAATAGCTAACATGCCAGCATCCACATTCTTTGCCATCATCTTCTTCCTCATGTTAATCACGCTGGGTTTGGATAGTACGTTCGCAGGCCTGGAAGGTGTGATCACAGCTGTGTTGGATGAGTTTCCTCACATCTGGGCCAAGCGCAGGGAATGGTTTGTGCTCATCGTGGTCATCACTTGCATCTTGGGATCCCTGCTCACACTGACATCAGGAGGGGCGTATGTGGTGACCCTGCTGGAGGAGTACGCCACGGGGCCAGCAGTGCTCACCGTGGCTCTCATCGAGGCCGTCGTCGTGTCTTGGTTCTATGGAATCACTCAGTTCTGCAGCGACGTGAAGGAAATGCTGGGCTTCAGCCCCGGATGGTTTTGGAGGATCTGCTGGGTGGCCATCAGCCCTCTGTTTCTCCTGTTCATCATTTGCAGTTTTTTGATGAGTCCACCCCAACTCCGGCTTTTCCAATACAATTATCCCCACTGGAGTATCATCTTGGGCTACTGCATAGGAACATCGTCTGTCATCTGCATCCCTATATACATCATTTATCGGCTGATCAGCACTCCAGGGACACTTAAGGAGCGCATTATTAAAAGTATCACTCCTGAAACACCAACGGAAATTCCGTGTGGGGACATCCGCATGAATGCTGTGTAACACACTGAGGGAGAGGACATGGCCTCCCAGCCCCCGACTCCTCATCTCTGAAAAGCCCCACCTTGACTCCTCCCCTCTAAGCCAAGCTGATGATGTAAGGTCTTTCTCCATGGAGTCACAGTCCTAAAGACTATGGTGCCCAGACTCTTGTGGGTTCCAACCACTTCTTTCCATGAACTCTCTTGGACTTACTGCCACATTAGCTGGTGACACGGCTGAGCTGACTTGGATATGTGAGGAGAGGAAGGAGGGGATGAACGCCACCCAGTCATCAGCTAGCTTCAGGTTTAGATTAGGTCTGTGAACGTCTGTATCATGTTCTGGGTATGATCATATTGCCCTGCATCTGTTTGCTTCTAAAGCCTTCAGTGTTCATGAATACATAAACCACCTAAGAGAAAACAGGGATGTCTTGCTAGCCATATATATTTTCTCAGTAGCATAGAACTCTGTAGCCGGAATCTACTAGAACCCTGTAACCCACGTGCTGCTGTGAGGTTAAGAAAGGAAGACGTAAAGACGCTACACTGAAAACTGATATATATGTGTGAGCTCTTGTGTCTGTCCATTGTTGTCTGTGTCCCCTCAATTCCAACACTCCGGGCCCATTACAAACTATATAAATGGCCTCTAATTTTTCTTACATTAAACAGATTCTACCTACTC

SEQ ID NO: 19 - DNA, 2747 bp, Rattus norvegicus; identical to SEQ ID NO: 17

SEQ ID NO: 20 - protein, 630 aa, Rattus norvegicus
METTPLNSQKVLSECKDREDCQENGVLQKGVPTTADRAEPSQISNGYSAVPSTSAGDEASHSIPAATTTLVAEIRQGERETWGKKMDFLLSVIGYAVDLGNIWRFPYICYQNGGGAFLLPYTIMAIFGGIPLFYMELALGQYHRNGCISIWRKICPIFKGIGYAICIIAFYIASYYNTIIAWALYYLISSLTDRLPWTSCTNSWNTGNCTNYFAQDNITWTLHSTSPAEEFYLRHVLQIHQSKGLQDLGTISWQLTLCIVLIFTVIYFSIWKGVKTSGKVVWVTATFPYIVLSVLLVRGATLPGAWRGVVFYLKPNWQKLLETGVWVDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHIWAKRREWFVLIVVITCVLGSLLTLTSGGAYVVTLLEEYATGPAVLTVALIEAVAVSWFYGITQFCSDVKEMLGFSPGWFWRICWVAISPLFLLFIICSFLMSPPQLRLFQYNYPHWSIVLGYCIGMSSVICIPTYIIYRLISTPGTLKERIIKSITPETPTEIPCGDIRMNAV

SEQ ID NO: 21 - DNA, 2162 bp, Gallus gallus
ggcaggagaaaccttggaaaaatggaaaataaggcaacgagcaatgagactcagccactgacttccaagaaaggtatctcagactgtaacgaaggagaagactgtaaagagaatggactgctgatcaggaaccctaaatctgccctacggctcgtggacgatgggaataaagtccatcctggacagggagataaagaggaggcagctcagatctcaaatggctattcaggagttcagagctccgggccttgcagtgggatgggagaggcggaggacgcgcagtgcactgccccagcagccacaaccaccaccacaaccaccacgtccaccacatgtggagcagaaggccagcagcagctgatggagctgggagacagagagacctggagtaaaaaaattgactttctgctctctgtcattggatatgcagtggatctgggaaacgtgtggagatttccttacatatgctatcagaatggaggaggagcattcctcattccttacacaattatggccatcttcggtggtatccctctcttctatatggaattagcattaggacagtaccacaggaatgggtgtatttcaatttggagaaaaatatgtcctatattcaaaggcattggctttgccatctgtataatcgacctctatgtagcctcctattacaacaccatcatggcgtgggtcttctactacctcgtgtcctccttcacgacggagctgccatggaccagctgcaataatgcatggaacacaggcaactgcactacctacttcagcaaggacaacatcagctgggccctgcactccatctctcctgcagaagaattttatacccgccaagttctacaggtgcataggtccaatgggctggatgacctggggggcattagctggcaattgaccctctgtttattgttaatcttcatcattgtatacttcagcatctggaaaggggtcaaaacatctggcaaggtggtgtgggtgacagccacgttcccttacgtcatactttttatcctgctcgtgagaggggcaactctgcctggtgcctggagaggtgtcctctactacctgaaacctgaatggcagaagctcctggccactgaggtttgggtggatgcagcagctcagatttttttctcccttggcccaggctttggggtcctattggcttatgccagctacaacaaattccataacaactgctaccaagacgccctggttaccagcactgtcaactgcctgaccagcttcgtgtccgggtttgtcattttcactgtgctgggatacatggccgagatgaggaacgaagatgtgtcggaggttgccaaagacatgggacccagtttgctcttcattacctatgccgaagccattgcgaacatgcctgcctcaactttctttgccatcatatttttcttgatgttactcacactgggattagacagcacgtttgcaggattagagggagtgattactggagtactggatgaattcccacacgtctggagcaagcgcagggaattctttgtcctcggtctgatcatcatttgctttttggggtcattagcaaccctgacttttggaggcgcatatgtggtgaagctgtttgaagaatacgccacaggcccagctgtgctaacagtggtgttcctggaagcggtggcagtggcttggttctacggcatcacccagttttgcaacgatgtgaaagaaatgctgggcttcgctccaggctggtactggcgagtgtgctgggtggcaatcagtcccatcttcctgctgtttgtcacttgcagctttctgtccaacccccccgagctccggctttttgattacaactatccctactggaccacagtagtgggctactgcataggaacctcttccatcatctgtatcccaatctacatggcttatcggttgatcatcactcctggaacacttaaggagcgtattctgaaaagcattactccagaaacagccacagaaattccttttggagacatccgcatgaatgccgtgtaagctaacctggtttctggggggagaggggaagcacagagtcaaactttccacccaaggaattatgtttccagctgagataatgggatgagttttctgcggaggttctgacactgacaacggacatttgg

SEQ ID NO: 22 - protein, 670 aa, Gallus gallus
MENKATSNETQPLTSKKGISDCNEGEDCKENGLLIRNPKSALRLVDDGNKVHPGQGDKEEAAQISNGYSGVQSSGPCSGMGEAEDAQCTAPAATTTTTTTTSTTCGAEGQQQLMELGDRETWSKKIDFLLSVIGYAVDLGNVWRFPYICYQNGGGAFLIPYTIMAIFGGIPLFYMELALGQYHRNGCISIWRKICPIFKGIGFAICIIDLYVASYYNTIMAWVFYYLVSSFTTELPWTSCNNAWNTGNCTTYFSKDNISWALHSISPAEEFYTRQVLQVHRSNGLDDLGGISWQLTLCLLLIFIIVYFSIWKGVKTSGKVVWVTATFPYVILFILLVRGATLPGAWRGVLYYLKPEWQKLLATEVWVDAAAQIFFSLGPGFGVLLAYASYNKFHNNCYQDALVTSTVNCLTSFVSGFVIFTVLGYMAEMRNEDVSEVAKDMGPSLLFITYAEAIANMPASTFFAIIFFLMLLTLGLDSTFAGLEGVITGVLDEFPHVWSKRREFFVLGLIIICFLGSLATLTFGGAYVVKLFEEYATGPAVLTVVFLEAVAVAWFYGITQFCNDVKEMLGFAPGWYWRVCWVAISPIFLLFVTCSFLSNPPELRLFDYNYPYWTTVVGYCIGTSSIICIPIYMAYRLIITPGTLKERILKSITPETATEIPFGDIRMNAV

SEQ ID NO: 23 - DNA, 2614 bp, Canis lupus familiaris
  misc_feature (2603)..(2603): n is a, c, g, or t
atggaaaccacagccttaaattcccagaaggagctatcagcatgtaaggataaagaagactgtcaggaaaatggagttctacagcagggtgtccctgtccccgaggataaggtggagtctggccaaatctccagtgggtactcagcagttccaagccctggtgcaggagatgaccctcagcactccattccagctgccaccacagccctagtggccgaagttcatcaaggggaacgagaggcctggggcaagaagatggatttcctcctgtcggtcattggctatgccgtggacctgggcaacgtctggcgcttcccctacatatgttaccagaatggagggggggcattccttctcccctacactatcatggccatttttggggggatcccgctgttctacatggagctcgcgctgggccagtaccaccgaaatggatgcatttcaatatggaggaaaatctgcccgattttcaaagggatcggttatgccatctgcatcattgccttctacattgcctcctactacaacaccatcatggcctgggcactctactacctcatttcgtccttcacggaccagctgccctggaccagctgcaagaactcctggaacactggcaactgcaccaactacttttccggggacaacatcacctggacgccccactcaacatcccctgcagaagaattttacatgcgccacgtcctgcagctccatcggtctaatggacttcaggacctgggtggcatcagctggcagctcaccctctgcatcatgctgatcttcgttgttatctacttcagtatctggaaaggtgtcaaaacatctggcaaggtggtatgggtgacagccaccttcccttatatcatcctttctgtcctgctggtaaggggggccaccctccctggagcctggaggggtgttctcttctacttgaaacccaactggcagaaactcctggagacaggggtgtgggtggatgctgctgctcagatcttcttctctcttggtcctggctttggggttttactggcttttgctagctacaacaaattcaacaacaattgttaccaagacgccctggtgaccagtgtggtgaactgtatgacgagcttcgtgtcaggatttgtcatcttcacagtgctcggatatatggctgagatgaggaacgaagatgtgtctaaggtggccaaagatgcaggccccagcctcctcttcatcacatatgcagaagccatagccaacatgccagcatccacattctttgctatcatcttcttcctgatgttgatcacactgggcctggacagcacttttgcaggcttggagggggtgatcacagctgtgctagatgagtttccacacatctggtccaagcgccgggagtggtttgtgctttgtgtggtcattacctgcttctttggatccctggtcaccctgacctttggcggggcatacgtggtgaagctgctggaggagtacgccacagggcccgcagtgctcaccgttgcacttatagaggcagttgctgtgtattggttttatggcatcactcagttctgcagtgacgtgaaggaaatgcttggcttcagccctggatggttctggaggatctgctgggtagccatcagccctctgtttctcctgttcatcatttgtagttttttgatgagcccaccacagctacgacttttccaatataattatccgcagtggagtatcatcctgggttactgcataggaacctcatctttcatctgcatccccacatatataacttaccggctggtcaccactccagggacatttaaagagcgtattattagaggcattactccagaaactgcgacagaaattccttatggggacatccatttgaatgccatataacatgcttcagtgagagaaagtggcttcctaacaatctcttcctctggttctgacaagtcatgcctcaccttcacccctctaagtgaatgagtttccagctacccctgacaatggaagggccttctcatggagagatggtcccagaagactatggtgcccagactcttatggcctccaatgacttgtttctctgaaatctcttggacatatcaccatgttaaatctatgacatagctaaactggactattttagatgtgtgaggctgtgtatggaggtcatgaaagccaccatctaatcaggattaggtttagaatcaagtttgtaaaagtctcctgcatcatttcttgttatgatcattggtatctgacatccatttgcttctaaaagtttcactgttcataaatgcataaaccatgtagaagaaaacagggacgctgttttgctagccatatattttctgagtagcatagaattctatagctggaatctactagaaccctgtaacaacccatgtgctgctgtggagtcaggaagatgtagaaactaaatggaaataatgtatacatgtgagcctgtgtatatgctgttgtttcagtggatccttatctctattctaatactttgggcccattacaaactatgaatttgctctgatttttcttacattaacaaatgtgtatgtatgttattataagggtataactactanaaaaaaaaaaa

SEQ ID NO: 24 - protein, 630 aa, Canis lupus familiaris
METTALNSQKELSACKDKEDCQENGVLQQGVPVPEDKVESGQISSGYSAVPSPGAGDDPQHSIPAATTALVAEVHQGEREAWGKKMDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSGDNITWTPHSTSPAEEFYMRHVLQLHRSNGLQDLGGISWQLTLCIMLIFVVIYFSIWKGVKTSGKVVWVTATFPYIILSVLLVRGATLPGAWRGVLFYLKPNWQKLLETGVWVDAAAQIFFSLGPGFGVLLAFASYNKFNNNCYQDALVTSVVNCMTSFVSGFVIFTVLGYMAEMRNEDVSKVAKDAGPSLLFITYAEAIANMPASTFFAIIFFLMLITLGLDSTFAGLEGVITAVLDEFPHIWSKRREWFVLCVVITCFFGSLVTLTFGGAYVVKLLEEYATGPAVLTVALIEAVAVYWFYGITQFCSDVKEMLGFSPGWFWRICWVAISPLFLLFIICSFLMSPPQLRLFQYNYPQWSIILGYCIGTSSFICIPTYITYRLVTTPGTFKERIIRGITPETATEIPYGDIHLNAI

SEQ ID NO: 25 - DNA, 2595 bp, Bos taurus
tcacctactagccagcaggatggagaccacccccttaaattcccagaaggagctatcagcgtataaggatggagaagattgtcaggaaaatggggttctacagaagggtgtccccgcccctggggataaggcagagtccggccagatctccaacgggtactcagcggttccgaaccctggcgcaggagacgacactcagcactccatcccggctgccaccaccgccctagtggctgaggttcatccagcagagcgggagacctgggccaagaaggtggatttcctcctctctgtcattggctatgctgtggacctgggcaacgtctggcgctttccctacatttgttaccagaatggagggggggcgttcctccttccctacactatcatggccatttttggggggatcccgctcttctacatggagctcgcactggggcagtaccaccgaaatggatgcatttcgatatggacaaaaatctgcccaattttcaaagggataggttgcgccatctgcctcatcgccttctacatcgcctcctactacaacaccatcatagcctgggccctctactacctcatctcctccttcacggagcagctgccctggaccagctgcgagaactcctggaacactggcaactgcaccaactacttctccgaggataacatcacctggacgctccactcaacgtcccctgcagaagaattttacacgcggcacgtcctgcagatccaccggtcgaaggggctccaggacctggggggcctcagttggcagcttgtcctgtgcatcatgttcatcttcactgttatctactttagcatctggaaaggcgtcaaaacatctggcaaggtggtttgggtgacagccaccttcccttacatcattcttttgatcctgctggtgaggggggccaccctccctggagcctggaggggagttctcttctatttgaaacccaactggcagaaactcctagagacgggggtgtgggtggatgcagccgcccagatcttcttctctctcggccctggctttggggtcctactggcttttgcgagctacaacaaattccacaacaactgttaccaagacgccctggtgaccagtgcagtgaattgcatgacgagcttcgtttcaggatttgtcatcttcacagtgctggggtatatggctgagatgaggaaagaagatgtgtctgaggtggccaaagatgcaggccccagcctcctgttcatcacatacgcagaagccatagccaacatgccagcatccacattctttgccatcgtcttcttcctgatgttaatcaccctgggcttggacagcacgtttgcaggcttggagggggtgatcacagctgtgctagatgagtttccacatgtctgggccaagcgccgggagtggtttgtgcttggcgtggtcattacctgcttctttggatctctggtcaccttgactttcggcggggcctatgtggtgaagctgttggaggagttcgccacgggacctgcagtgctcaccgtggccctgattgaagcagttgctgtgttttggttctacggcatcaatcagttctgcagtgatgtgaaggaaatgcttggtttcagccctggatggttctggaagatctgctgggtagccatcagtcctctgtttctcctgttcatcatctgcagttttttgatgagcccaccacagctacgacttttccagtatgattatcctcggtggagcatcatcctgggttactgcataggaacctcatctttcatctgcatccccacatatataacctatcggctgattgtcactccagggacacttaaggagcgtattattaaaggtatcacgccagaaacaccgacagcaattccctgtggggacatccgcttgaatgctgtgtaaaacatcaacaaaaggaagatggcttcccaatacctcttcctccagtcctgacaagtcacacatcgccttctccctctaagtcaatgagtttccggctaagcctatcaatggaagggccttcttcatagagacacagtcccaaaagactacggtgcccagattttttcggcctccagtcactttgtgtgaaatctccgggacatgttaccacgttagactctgtgatgcagctaaagtggactgctttgaatgtgtgaagctgtgtgtgaaaaccaccatgtcatcattaggactagtttaggatcaagtctgtgaaagtctcctatgtcattccttcttatgatcatcagtatctaatattgtttgcttccaaagatttcaactgctcatgactgcataaaccatgtaggaaaaaacagggatactgtcttgctagccatatgttttctgagtagcagagaatcctatagctggagtctattagaaccctgtaacctgtgtgctgctgtggaatcaggagaggaagatttaagaagcttaaattgaaaaaataatgtgtatgtgtgagcatgtgtgtctgtgtgtacaactgttccagtgtatccttacccattacaaactatatgaattcctctagtttttttttacattaacaaattccacctactaaaaaaaaaaaaaaa

SEQ ID NO: 26 - protein, 630 aa, Bos taurus
METTPLNSQKELSAYKDGEDCQENGVLQKGVPAPGDKAESGQISNGYSAVPNPGAGDDTQHSIPAATTALVAEVHPAERETWAKKVDFLLSVIGYAVDLGNVWRFPYICYQNGGGAFLLPYTIMAIFGGIPLFYMELALGQYHRNGCISIWTKICPIFKGIGCAICLIAFYIASYYNTIIAWALYYLISSFTEQLPWTSCENSWNTGNCTNYFSEDNITWTLHSTSPAEEFYTRHVLQIHRSKGLQDLGGLSWQLVLCIMFIFTVIYFSIWKGVKTSGKVVWVTATFPYIILLILLVRGATLPGAWRGVLFYLKPNWQKLLETGVWVDAAAQIFFSLGPGFGVLLAFASYNKFHNNCYQDALVTSAVNCMTSFVSGFVIFTVLGYMAEMRKEDVSEVAKDAGPSLLFITYAEAIANMPASTFFAIVFFLMLITLGLDSTFAGLEGVITAVLDEFPHVWAKRREWFVLGVVITCFFGSLVTLTFGGAYVVKLLEEFATGPAVLTVALIEAVAVFWFYGINQFCSDVKEMLGFSPGWFWKICWVAISPLFLLFIICSFLMSPPQLRLFQYDYPRWSIILGYCIGTSSFICIPTYITYRLIVTPGTLKERIIKGITPETPTAIPCGDIRLNAV

SEQ ID NO: 27 - DNA, 2340 bp, Cavia porcellus
ttactaaccagcaggatggagaccacagccttgaattctcagaaggcgccgtcggtgtgtaaggacagagaggattgtcaggaaaacagcattttacagaagagtggtcctacatcggcgggtggagtggagtctggccagatattcaatgggtactcatcggttcccagcactggcatgggggatgacgctgagcactctgtcccgactgccaccaccaccctggtggctgaggtgcaccatggggagcgggagacctggggcaagaaggtagacttcctcctctcggtcatcggctacgctgtggacctgggcaacatctggcgctttccctatgtgtgttaccagaacggtggaggagcattcctcctcccctacatcatcatggccatctttggagggatcccactgttttacatggagcttgcgttgggccagtatcaccggaatggatgcatttctatatggaggaaaatctgcccgatttttaaagggattggctacaccatctgcatcatcgccttctacattgcctcctactacaacaccatcatagcctgggccctctactacctcatctcttcctttacggaccggctgccctggaccagttgcaggaactcctggaacacggccaactgcaccaactacttctctgaggacaacatcacctggacgctccactccacctcccctgcggaagagttttatatacgccacatccttcagatccatcggtctaagggactgcaggacgtgggaggcgtcagttggcagcttactctctgcatcatgcttatctttaccatcatatacttcagcatctggaaaggtgttaaaacatctggcaaggtggtgtgggtgacagctacctttccttatatcgtcctttctgtgctgctggtgaggggggccacactcccaggagcgtggaagggtgttctcttctacttgaaacccaactggcagaagctcctggagacaggggtgtggattgatgctgctgctcagatattcttctctcttggtccaggctttggggtcctcctggcctttgccagctataacaagttcaacaacaactgttaccaagatgccctggtgaccagtgcggtgaattgcatgacgagcttcgtttcggggtttgtgatcttcacggtactggggtacatggctgagatgaggagcgaagatgtatctgaagtagccaaagatgcaggccccagcctcctcttcatcacctatgcagaggccatagccaacatgccggcatccacgttttttgccatcatcttcttcctcatgttaatcacactgggtctggacagcacgtttgcaggcttggagggagtgatcactgcagtgctggatgagtttccacacatctgggccaagcaccgggagtggtttgtgcttgctgtggtcattacttgcttctttggatccctgaccaccctgacttttggaggggcctacgtggtgaagttgctggaggagtacgccacggggcctgcggtgctcaccgtggtgttcatagaagcaatcgctgtgtcttggttctatggggtcactcagttctgcagcgatgtgaaggagatgctcggcttcagcccgggatggttttggaggatctgctgggtggcggtcagccctgtgttcctcctgttcatcatctgcagttttctgatgagcccaccccagctacgacttttccaatacagttatccccactggagtgtcatcctgggttactgcatagggacttcctccgtcatctgcatccccacatacattacctaccggctggtcaccaccccagggaccctgaaggagcgcattattaaaagtatcactccagaaacaccaacagaaattccttgtggggatatctgcttgaatgctgtgtaacacattcctgtgagaaacgattcccagccctccagctctgacgaatcccacctcacctccgctcaaagtgaatcagtttccagccaagcctgatgatgaaaaggatcttctttgcggagacacagtcccaaaagactaatggtgcccagactacggcttccagtcacttcctcccattaaatctctgggacattatcactatgttagactatgatgcaactgaagtggcctattttgaacgtgtgagggtgtgtatggaggtgatgaaaactaccctatcattgattaggattaggtttagaatcaagtttgtggatgtttcccgtgtcatttttttggtatgactgttatctgacatttgtttgcctctaaaggcttcactgttcatgaatgcattaaacattagaagaaaataaaaaaaaaaaaaaaaaa

SEQ ID NO: 28 - protein, 630 aa, Cavia porcellus
METTALNSQKAPSVCKDREDCQENSILQKSGPTSAGGVESGQIFNGYSSVPSTGMGDDAEHSVPTATTTLVAEVHHGERETWGKKVDFLLSVIGYAVDLGNIWRFPYVCYQNGGGAFLLPYIIMAIFGGIPLFYMELALGQYHRNGCISIWRKICPIFKGIGYTICIIAFYIASYYNTIIAWALYYLISSFTDRLPWTSCRNSWNTANCTNYFSEDNITWTLHSTSPAEEFYIRHILQIHRSKGLQDVGGVSWQLTLCIMLIFTIIYFSIWKGVKTSGKVVWVTATFPYIVLSVLLVRGATLPGAWKGVLFYLKPNWQKLLETGVWIDAAAQIFFSLGPGFGVLLAFASYNKFNNNCYQDALVTSAVNCMTSFVSGFVIFTVLGYMAEMRSEDVSEVAKDAGPSLLFITYAEAIANMPASTFFAIIFFLMLITLGLDSTFAGLEGVITAVLDEFPHIWAKHREWFVLAVVITCFFGSLTTLTFGGAYVVKLLEEYATGPAVLTVVFIEAIAVSWFYGVTQFCSDVKEMLGFSPGWFWRICWVAVSPVFLLFIICSFLMSPPQLRLFQYSYPHWSVILGYCIGTSSVICIPTYITYRLVTTPGTLKERIIKSITPETPTEIPCGDICLNAV